(19) United States
(12) Patent Application Publication
(10) Pub. No.: US 2017/0368182 A1
(43) Pub. Date: Dec. 28, 2017

(54) Pharmaceutical Semi-Solid Gels with Entrapped Calcium Phosphate Nanoparticles

(71) Applicant: Belmont University, Nashville, TN (US)

(72) Inventor: Kenneth Reed, Nashville, TN (US)

(21) Appl. No.: 15/534,930

(22) PCT Filed: Dec. 9, 2015
(86) PCT No.: PCT/US15/64817

§ 371 Date: Jun. 9, 2017

Related U.S. Application Data
(60) Provisional application No. 62/089,234, filed on Dec. 9, 2014.

(51) Int. Cl. A61K 47/38; A61K 47/02; A61K 33/06; A61K 9/10; A61K 33/42; C01B 25/32; A61K 9/14

(52) U.S. Cl. CPC A61K 47/38; C01B 25/32; A61K 47/02; A61K 9/14; A61K 9/10; A61K 33/42; A61K 33/06; A61K 2300/00; A61K 2800/413

(57) ABSTRACT

The present disclose generally provides pharmaceutical semi-solid hydrogels with entrapped calcium phosphate Comparison of Fitted Curves with Log Time for nanoparticles that demonstrate enhanced Preparations Demonstrating Statistical Significance drug release, retention, and esthetic properties. The hydrogels are particularly useful for topical applications of drug molecules. The present disclosure also relates to methods of administering a pharmaceutical agent by providing a pharmaceutical semi-solid hydrogel containing at least one pharmaceutical agent and administering it to a subject in need thereof.

### CROSS-REFERENCE TO RELATED APPLICATIONS

This application claims the benefit of priority to U.S. Provisional application No. 62/089,234 filed on Dec. 9, 2014, the contents of which are hereby incorporated by reference in their entirety.

### TECHNICAL FIELD

The present disclose generally relates to pharmaceutical semi-solid hydrogels with entrapped calcium phosphate nanoparticles that demonstrate enhanced drug release, retention, and esthetic properties. The hydrogels are particularly useful for topical application of drug molecules.

### BACKGROUND ART

Ophthalmic beta-blockers are commonly used in the initial treatment of open-angled glaucoma. The use of low viscosity ophthalmic Timolol maleate solutions results in rapid removal of drug product from the precorneal area. Normally less than 5% of Timolol actually reaches intraocular tissues. These characteristics result in the frequent dosing recommendation of twice daily. Drug absorption occurs via the nasolacrimal drainage duct after drainage of Timolol from the precorneal area, which in turn leads to an increased likelihood of systemic absorption. Resultant systemic CV beta blocking effects may cause bradycardia, heart block, heart failure, and asthma. Thirty two deaths were reported for the first seven years use of Timolol maleate. An ophthalmic drug delivery system with a decreased drug release rate may lead to less frequent dosing, fewer adverse drug events, lower required drug concentrations, better tolerability, and improved patient compliance with ophthalmic drug products, such as timolol. Ophthalmic gels may allow for an improvement in ocular drug delivery by lengthening the residence time of the drug in the eye (increased corneal contact) and decreasing the amount of drug absorbing into the systemic circulation. These properties are attributed to the higher viscosity of gels as compared to solutions. Drug release from gels is also expected to be slowed as compared to a solution dosage form. These properties would be advantageous in other topical drug formulations as well.

Dibasic calcium phosphate dihydrate [7789-77-7] has been used as a tablet and capsule diluent for a number of years (Pharmaceutical Excipients, 3^{rd} ed. © 2000 American Pharmaceutical Association and Pharmaceutical Press. CD-ROM). Calcium phosphates are useful as pharmaceutical tablet fillers and binders due to their good binding properties, flowability, low cost, chemical purity and compatibility with pharmaceutical drugs.”

Calcium phosphates that are useful in pharmaceutical tableting include dibasic calcium phosphate dihydrate (CaHPO₄.H20; DCP or DCPD), dibasic calcium phosphate anhydrous (CaHPO₄; DCPA or ACP) and hydroxyapatite (Ca₅(PO₄)₃OH″; HP or HAP or HA). (Calcium phosphates in pharmaceutical tableting. (Physico-pharmaceutical Propeties by P. C. Schmidt and R. Herzog. Pharmacy World & Science, 15(3):105-115, 1993.)

Dibasic calcium phosphate dihydrate is generally regarded as a nontoxic and nonirritant material (GRAS listed). It is included in the FDA Inactive Ingredients Guide (oral capsules and tablets) and in non-parenteral medicines licensed in Europe, the UK, and the US. However, oral ingestion of large quantities may cause abdominal discomfort.

The calcium phosphates are insoluble in water as defined by the USP. (Physical and Chemical Properties of Calcium Phosphates for Solid State Pharmaceutical Formulations by J. R. Carstensen and C. Ertell. Drug Development and Industrial Pharmacy, 16(7):1121-1133, (1990).) Dicalcium phosphate is only soluble at low pH values and is insoluble at physiological pH (0.002 g in 100 gm of water). In general, the calcium phosphates become increasingly soluble below pH environments that are less than 6.5. (Calcium Phosphate Nanocomposite Particles for In Vitro Imaging and Encapsulated Chemotherapeutic Drug Delivery to Cancer Cells by Mark Kester et.al., Nano Letters, 8 (12):4116-4121, (2008).)

Calcium phosphates are also a major component of bone and tooth enamel, where it is seen in the form of amorphous calcium phosphate (ACP) as well as crystalline hydroxyapatite (HAP), the major component of bone and tooth enamel. Additionally, both Ca²⁺ and PO₄³⁻ are found in relatively high concentrations at typically 1-5 mM in the bloodstream. Encapsulation of Organic Molecules in Calcium Phosphate Nanocomposite Particles for Intracellular Imaging and Drug Delivery by Thomas T. Morgan et.al., Nano Letters, 8 (12):4108-4115, (2008).) As a biomineral, CP safely biodistributes, with dissolved material regulated via the kidneys. CP is relatively insoluble at physiological pH but has increasing solubility in the acidic environments that can occur in the body, such as in endolysosomes. It is suggested that calcium phosphate nanoparticles dissolve when the endosomes carrying them fuse with lysosomes where they experience low pH.

HP and DCP were found, when suspended in saturated saline water, to give pH values of 6.6 and 6.0, respectively. The zeta potential for HP was measured to be about +13 mV in a pH 6.6 medium with an ionic strength of 0.154. The zeta potential for DCP was measured to be about +3 mV in a pH 6.0 medium with an ionic strength of 0.154. (Effect of Two New Polysaccharides on Growth, Agglomeration and Zeta Potential of Calcium Phosphate Crystals by E. R. Boeve et.al. The Journal of Urology, 155:368-373, 1996.) The zeta potential is defined as the difference in potential between the surface of a tightly bound layer (called the shear or slipping plane) and the electro neutral region of the solution.

Presumably, the positively charged HP and DCP particles decrease the pH of the saline solution by strongly absorbing OH^{—} groups to the slipping plane. Nanometer size particles are frequently difficult to disperse, especially at high ionic strength. Presumably, the low zeta potential facilitates the formation of these agglomerates.

The zeta potential for hydroxyapatite (HA) was measured as −2 to −10 mV in pure water. This was increased to a value of about −45 to −50 mV by adsorbing sodium citrate onto the HA particles. This in turn resulted in a decrease of the attractive inter particulate forces and a measured decrease of dilatant flow behavior. (Ionic modification of calcium phosphate cement viscosity. Part I:hypodermic injection and strength improvement of apatite cement by Uwe Gbureck et.al., Biomaterials, 25:2187-2195 (2004). It can be assumed that the citrate absorption onto HA is due to the presence of citrate carboxylic groups.

The zeta potential for dicalcium phosphate anhydride (DCPA) in pure water was measured as −15 to −18 mV in pure water. As seen below, the zeta potential increased in the presence of sodium phosphate and with increasing pH. The addition of antibiotics resulted in zeta potential changes to both DCPA and hydroxyl apatite (HA), which indicates that binding to the surface was taking place. (²⁰Surface properties of calcium phosphate particles for self setting bone cements by U. Gbureck et.al., Biomolecular Engineering, 19:51-55 (2002).)

In agreement with previously cited work, the zeta potential for calcium titanium phosphate particles (CTP) and hydroxyapatite particles (HAP) increased with increasing pH. (Calcium phosphate-alginate microspheres as enzyme delivery matrices by C C. Ribeiro et.al., Biomaterials, 25:4363-4373 (2004).)

Hydrogels are three-dimensional, cross-linked networks of water-soluble polymers. Drugs can be loaded into the gel matrix due to porosity of the gel, and subsequent drug release occurs at a rate dependent on the diffusion coefficient of the small molecule or macromolecule through the gel network. A depot formulation is created from which drugs slowly elute, maintaining a high local concentration of drug in the surrounding tissues over an extended period. Biocompatibility is promoted by the high water content of hydrogels. Therefore, it can be seen that hydrogels are an advantageous dosage form, especially for ocular administration.

In general, the rate of drug release from a linear polymer matrix is inversely proportional to its viscosity. (Hydrogels in drug delivery: Progress and challenges by Todd R. Hoare and Daniel S. Kohane, Polymer 49:11993-2007 (2008).) This causes a difficulty in that very large unworkable viscosities may be needed to affect a desired prolonged release. Water-soluble polymer hydrogels that are not cross-linked swell and subsequently dissolve in the aqueous in vivo environment. (Hydrogels in drug delivery: Progress and challenges by Todd R. Hoare and Daniel S. Kohane, Polymer 49:11993-2007 (2008).) Thus, if the polymers can be cross linked, it is likely they will stay longer in an in vivo environment such as the area of the eye.

Cross-links between the different polymer provide networks that have visco-elastic and sometimes pure elastic behavior. Polymers can be cross linked physically in addition to chemically. Alginate, for example, can be cross linked by ionic interactions, such as through calcium ions. “In addition to anionic polymers being cross linked with metallic ions, hydrogels can also be obtained by complexation of polyanions with polycations. lonically crosslinked chitosan hydrogels are formed by complex formation between chitosan and polyanions, such as dextran sulfate or polyphosphoric acid.” (Novel Crosslinking methods to design hydrogels by W. E. Hennink and C. R. van Nostrum, Advanced Drug Delivery Reviews 64:223-236 (2012).)

Control release tablets of the poorly water soluble drug Ibuprofen were fabricated and tested for Ibuprofen release. “Carbopol highly swells when exposed to pH environment above its pKₐ of 6±0.5. The rapid gel formation may be due to the ionization of carboxylate group, resulting in repulsion between the negative particles that adds swelling of polymer. This swelling is thought to be responsible for controlling the release of drug.” (Controlled Release Oral Delivery System Containing Water Insoluble Drug by Panna Thapa et.al., Kathmandu University Journal of Science, Engineering and Technology 1(1):1-10 (2005).) The release studies demonstrated that as the carbopol concentration increased (with a concomitant decrease in DCP tablet concentration) the rate of release became slower and more linear. The authors stated that “The reason for this could be that the gel layer formed around the tablet becomes stronger, with few interstitial spaces between the microgels.”

In summary, it would useful to provide a hydrogel formulation with improved viscosity, tortuosity and drug release characteristics, as well as with improved aesthetics, in order to provide a pharmaceutical carrier compositions. Such compositions would be useful for providing various pharmaceutical formulations, and particularly topical formulations.

### SUMMARY OF THE INVENTION

The present disclosure provides a pharmaceutical composition in which calcium phosphate nanoparticles are added to a hydrogel in order to increase the tortuosity and viscoelastic properties of the gel. This is anticipated to result in a more prolonged and controllable sustained release of active drug molecules. Additionally, the gel should be transparent and thus be more acceptable as a topical dosage form (especially as an ophthalmic dosage form). The gel described here should be more aesthetically pleasing for gels that are applied in topical locations other than the eye (e.g., topically to the skin). More particularly, such compositions may be particularly advantageous for ophthalmic uses. Such formulations also could be useful for other modes of administration, including oral, topical to the skin (dermal), rectal and vaginal application.

Accordingly, the present disclosure provides a pharmaceutical composition comprising a hydrogel and calcium phosphate nanoparticles. In particular embodiments, the hydrogel comprises polymers having functional groups selected from the group consisting of carboxylate, carbonyl, amine and mixtures thereof. Examples of hydrogels useful in the present disclosure include, but are not limited to, hyaluronic acid, alginate, carbopol (cross-linked polyacrylate), sodium alginate, carboxymethyl cellulose, and mixtures thereof.

Calcium phosphate nanoparticles have a size ranging from about 5 to about 200 nm, and more particularly, from about 10 to about 100 nm, and even more particularly, from about 10 to about 80 nm.

In some embodiments, the composition comprises about 0.1% to about 5% of the calcium nanoparticles by weight of the composition.

The present disclosure further provides a method of administering a pharmaceutical agent to a patient in need thereof comprising providing a pharmaceutical composition comprising a hydrogel, calcium phosphate nanoparticles, and a pharmaceutical agent, and administering the composition to the patient.

It is to be understood that both the foregoing general description and the following detailed description present embodiments of the disclosure and are intended to provide an overview or framework for understanding the nature and character of the disclosure as it is claimed. The description serves to explain the principles and operations of the claimed subject matter. Other and further features and advantages of the present disclosure will be readily apparent to those skilled in the art upon a reading of the following disclosure.

### BRIEF DESCRIPTION OF THE DRAWINGS

FIG. 1 is a graph of a timolol maleate standard absorbance curve at 295 λ at different concentrations. The equation was not forced to a Y-intercept of zero and had an r² value of 0.9999. Concentrations of timolol maleate in experimental samples were determined using this standard absorbance curve.

FIG. 2 is a graph depicting a representative sample of a typical timolol release profile from a 1% carbopol 980 gel having a sigmoidal fit, along with the individual data points (85 pairs.

FIG. 3 is a graph of a typical timolol release profile as in FIG. 2, where the fit and data are shown with log (time) rather than time as the X-axis.

FIG. 4 is a graph depicting one approach to examining the linearity of release, in which the linear portion of the curve examined as a measure of the rate of release. The initial data pairs were fit using linear regression.

FIG. 5 is a graph showing the comparison of initial linear and sigmoidal fit for timolol release another approach to examining the linearity of timolol release.

FIG. 6 is a graph depicting the linear character of the sigmoidmal fit for representative timolol-gel-calcium phosphate nanoparticle formulations.

FIG. 7 is a graph that plots the first derivative of the difference from linear values versus time for representative timolol-gel-calcium phosphate nanoparticle formulations.

FIG. 8 is graph depicting the comparison of fitted curves for timolol release from representative calcium phosphate nanoparticle preparations.

FIG. 9 is graph depicting the comparison of fitted curves for timolol release in log time from representative calcium phosphate nanoparticle preparations.

### DETAILED DESCRIPTION

Reference now will be made in detail to the embodiments of the present disclosure, one or more examples of which are set forth herein below. Each example is provided by way of explanation of the compositions of the present disclosure and is not a limitation. In fact, it will be apparent to those skilled in the art that various modifications and variations can be made to the teachings of the present disclosure without departing from the scope or spirit of the disclosure. For instance, features illustrated or described as part of one embodiment, can be used with another embodiment to yield a still further embodiment.

Thus, it is intended that the present disclosure covers such modifications and variations as come within the scope of the appended claims and their equivalents. Other objects, features and aspects of the present disclosure are disclosed in or are obvious from the following detailed description. It is to be understood by one of ordinary skill in the art that the present discussion is a description of exemplary embodiments only and is not intended as limiting the broader aspects of the present disclosure.

While not being bound by any particular theory, the addition of calcium phosphate nanoparticles in the present hydrogels is expected to increase the tortuosity and viscoelastic properties of the gel. This is anticipated to result in a more prolonged and controllable sustained release of active drug molecules. Additionally, the gel should be transparent and thus be more acceptable as a topical dosage form (especially as an ophthalmic dosage form). The gel described here should also be more aesthetically pleasing for gels that are applied in locations other than the eye (e.g., dermatologically). It is anticipated that this type of gel could also be administered orally.

The present disclosure provides a pharmaceutical composition comprising a hydrogel and calcium phosphate nanoparticles. The present pharmaceutical composition are useful as carriers for pharmaceutical agents. Selection (Surface Interactions) of Calcium Phosphate:

Calcium phosphate (CaP) particles were shown in the literature to readily interact with carboxylate, carbonyl, and amine functional groups. This interaction was of sufficient intensity to be referred to as forming bonds and coordination complexes. Although not an exhaustive listing, a review of the listed literature indicates that calcium phosphate surface interactions were found to occur with antibiotics, collagen, heparin analogues, sodium citrate, bovine serum albumin, both types of gelatin, proteins, hyaluronic acid, alginic acid, DNA, and a carbonic acid inhibitor. While not being bound by theory, it appears that CaP will readily undergo an electrostatic type of surface interaction with a low or high molecular weight molecule as long as the correct functional groups are available. Considering the wide ranging adsorptive properties of calcium phosphate particles, the listed functional groups are not exclusively needed in order for a CaP NP-polymer interaction to occur. Rather, the listed functional groups should be considered as favoring the formation of a CaP NP-polymer interaction.

### Selection of Form of Calcium Phosphate

The number ratio of C to P in the calcium phosphate nanoparticles in some embodiments ranges from 1:1 to 3:1. In an embodiment, the form of calcium phosphate is dibasic calcium phosphate dihydrate (CaHPO₄.H20). In another embodiment, the form of calcium phosphate is tricalcium diphosphate (Ca₃(PO₄)₂) because it is readily available in nanometer size. Favored forms of calcium phosphate include dibasic calcium phosphate anhydrous (CaHPO₄) and hydroxylapatite (Ca₅(PO₄)₃OH) because of their common pharmaceutical use. All Calcium Phosphates (chemical bond between calcium and phosphorus atoms, the phosphorus atom is further bonded to oxygen atoms that may or may not be bonded to hydrogen) that are insoluble at pH of 6.5 to 8.0 are considered to be appropriate for the pharmaceutical compositions described herein

### Selection (Safety) of Calcium Phosphate Nanoparticles:

Calcium phosphates have a long history of being safely used in pharmaceutical products and are present normally in the body, and it is generally agreed that Calcium Phosphate Nanoparticles (CaP NP) are safe. If CaP NPs are ingested by cells, they are soluble in endolysosomes and should be converted to Ca and P, elements that are present endogenously in the body. Selection (physical properties) of Nanoparticles:

Discrete nanoparticles are expected to be transparent when dispersed in water. This will result in less blurred vision when administered to the eye. Additionally, it should be more aesthetically pleasing if used as a dermatological dosage form. If the dermatological is used as a film forming bandage, the transparency would enable inspections of wounds and abrasions without removing the protective film (bandage).

In some embodiments, the calcium phosphate nanoparticles have a size ranging from about 5 nm to about 200 nm. In other embodiments, the calcium phosphate nanoparticles have a size ranging from about 10 nm to about 100 nm, while in still other embodiments, the calcium phosphate nanoparticles have a size ranging from about 10 to about 80 nm.

The calcium phosphate nanoparticles useful in the present compositions have an increased surface area compared to stand calcium phosphates used in the pharmaceutical industry. For example, in some embodiments, the calcium phosphate particles have a surface area ranging from about 10 m²/gm to about 100 m²/gm, while in other embodiments, the calcium phosphate particles have a surface area ranging from about 30-60 m²/gm.

Indeed, nanoparticles have a huge increase in surface area as compared to particles in the micron range or larger (see below). The surface area for nanoparticles of tricalcium diphosphate (10-80 nanometers) is given by the vendor as 30-60 m²/gm as compared to the surface area of 0.44-0.46 m²/gm given for a commercial tableting grade of dibasic calcium phosphate dehydrate¹. This increase in surface area should magnify the surface interactions anticipated between CaP and suitable hydrophilic polymers.

### Selection of Hydrophilic Polymers:

Polymers used to form the hydrogels are preferably water soluble and in some embodiments possess carboxylate, carbonyl, and/or amine functional groups. The carboxylate group appears to give the strongest interactions with CaP. While not being bound by theory, it is believed that the use of solid particles of CaP should prevent the polymer precipitation sometimes seen with high concentrations of aqueous Ca⁺² ions. The most favorable hydrophilic polymer for this use is hyaluronic acid. It is a natural high molecular weight polymer with a significant number of carboxylate groups that is biodegradable and has been extensively used in eye cataract surgery. In other embodiments, hydrophilic polymers are carbopol, sodium alginate and carboxymethyl cellulose. Such polymers are useful due to their high molecular weights, abundant carboxylate groups, and excellent safety profiles. Other water soluble (non-ionic) polymers, are also useful in some embodiments.

### Importance of Tortuosity (or Porosity)

Tortuosity is understood in the art as referring to a property of a curve being tortuous (twisted; having many turns). There have been several attempts to quantify this property. Tortuosity is commonly used to describe diffusion in porous media. It is commonly invoked in hydrogels to explain why the release of drug molecules is slowed when dissolved in a hydrogel network. That is, the drug molecule must work its way through the polymer network in order to be released out of the system. The use of CaP NPs will result in multitudes of NPs blocking the pathway of the drug molecule, thus slowing its release even more. Additionally, most drug particles would adsorb onto CaP NP and the need to desorb would result in additional slowing of their release from the hydrogel.

### Importance of the Hydrophilic Polymer Ensuring Dispersed Nanoparticles:

It is anticipated that just as adsorbing Na citrate onto CaP NP resulted in dispersions, that the same dispersive events will occur when hydrophilic polymers adsorb onto CaP NPs. Agglomerations of the CaP NPs would result in a loss of transparency and significantly decrease the overall strength of the interactions between solid phase and solvated polymer.

### Importance of Increasing the Viscoelastic Properties of the Resulting Hydrogel:

It is anticipated that the binding of CaP NPs to hydrophilic polymer strands will result in a dramatic increase in viscosity (resistance to flow) and elasticity (resistance to deformation) of the resulting hydrogel. Examples were given in the literature where by physical crosslinking of polymers resulted in such effects.

An increase in viscosity would resist tear flow in the eye and increase retention time of an ophthalmic gel. An increase in elasticity may increase ocular retention time by absorbing the energy of blinking, much as a contact lens does. An increase in elasticity may assist in setting up a dermatological gel so that it stays in place until a film forms from evaporation. An increase in elasticity could help to insure that a capsule filled with such a gel would empty out of the stomach intact and then break into micro hydrogels in the gastro intestinal tract.

Increases in viscosity should slow down the release of drug that is either suspended or dissolved in the CaP-Polymer hydrogel. It of note, that the release rate can be controlled by the amount of either CaP or polymer and the ratio between them. Thus, an increased flexibility in design of the products release rate is achieved.

The pharmaceutical compositions described above can be used as carriers or delivery vehicles for a variety of pharmaceutical agents.

The pharmaceutical agent useful with the present pharmaceutical compositions is not limited, and any pharmaceutical may be used provided it is stable in and compatible with the hydrogel and calcium phosphate nanoparticles used in the composition. For example, low molecular weight active ingredients (small molecules) such as, for example, many antiviral substances, hepatotherapeutic substances, neuroprotective substances, immunotherapeutics and -suppressants, low molecular weight active ingredients for cardiovascular disorders or cancer, analgesics, low molecular weight antiinflammatory, antibiotic and antimicrobial active ingredients, beta-blockers (such as timalol) and low molecular weight hormones, or macromolecular active ingredients such as, for example, nucleic acid fragments or nucleic acids (genomic DNA, cDNA, mRNA, siRNA, antisense oligonucleotides etc.). Examples of low molecular weight active ingredients are nucleoside analogues, beta-interferons, alpha lipoic acid, peptide analogues, enzyme or receptor inhibitors, agonists and antagonists, prostaglandins, steroids, cytostatics and heterocyclic antibiotics. It is also possible in particular to employ active ingredients in the form of their prodrugs.

Examples are provided to illustrate some embodiments of the compositions of the present disclosure but should not be interpreted as any limitation thereon. Other embodiments within the scope of the claims herein will be apparent to one skilled in the art from the consideration of the specification or practice of the compositions or methods disclosed herein. It is intended that the specification, together with the example, be considered to be exemplary only, with the scope and spirit of the disclosure being indicated by the claims which follow the examples.

### EXAMPLES

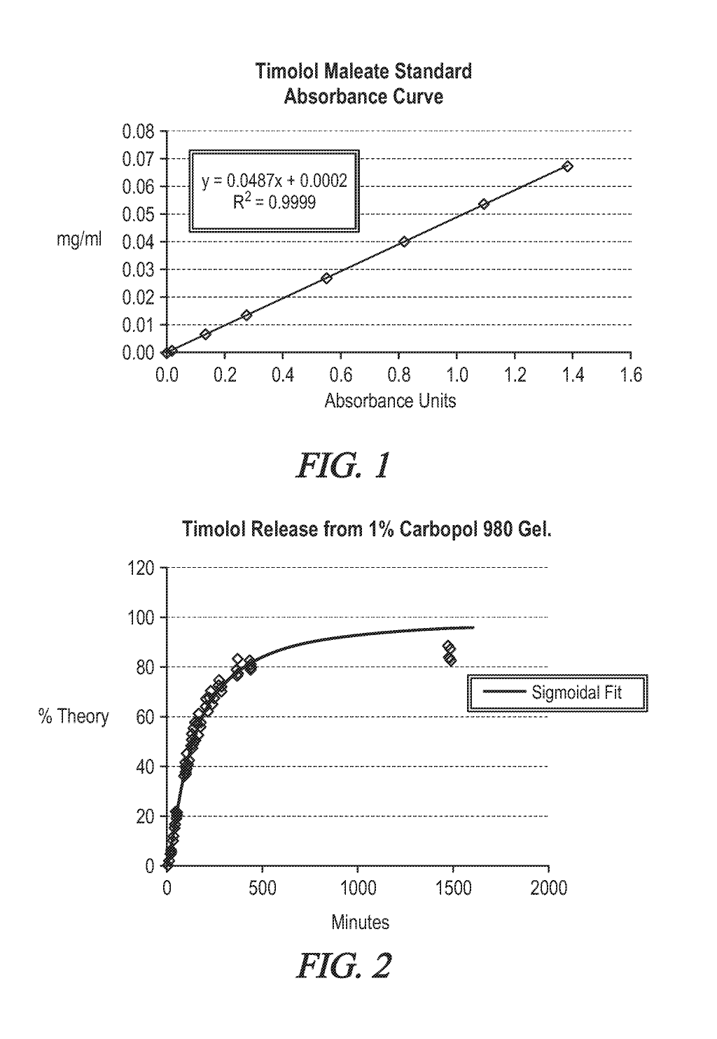

A UV-Visible Spectrophotometer was used to measure the amount of UV absorbance at 295 λ of Timolol maleate standard solutions at eight different concentrations. A calibration curve was then generated between Timolol maleate absorbance at 295 λ and concentration (FIG. 1). The equation was not forced to a Y-intercept of zero and had a r2 value of 0.9999. Concentrations of Timolol in experimental samples were determined using the standard curve.

### Example 1

### Timolol Preparation in a Carbopol Hydrogel with Calcium Phosphate Nanoparticles

Open-angle glaucoma is an eye disease defined as an optic neuropathy that results in the atrophy of the optic nerve head and is associated with loss of vision. Increased intraocular pressure (IOP) may be present in patients with glaucoma. Pharmacologic therapies to treat glaucoma focus on lowering IOP. A clear fluid called aqueous humor maintains IOP. The front of the human eye is filled with aqueous humor and it is continually produced by the ciliary body. In addition to maintaining IOP, aqueous humor protects and provides nutrients for the eye. Aqueous humor is drained from the eye through the nasolacrimal duct into the patient's systemic circulation. Glaucoma often results from blocked drainage of aqueous humor, resulting in increased IOP. Lowering IOP can be accomplished by either increasing the outflow of aqueous humor or by decreasing production of aqueous humor. According to the American Academy of Ophthalmology Glaucoma guidelines, ophthalmic prostaglandin analogs and ophthalmic beta-blockers are both considered first-line therapy in the initial treatment of open-angled glaucoma.³ Prostaglandin analogs increase aqueous humor outflow and have been shown to have superior decrease in IOP compared to beta-blockers. The use of prostaglandin analogs is primarily limited due to the adverse effects of brown discoloration of the iris and lengthening and darkening of eyelashes. These adverse effects are particularly a problem in patients with glaucoma in one eye. Ophthalmic beta-blockers suppress aqueous humor production and avoid the cosmetic adverse effects caused by prostaglandin analogs. The main limitations of ophthalmic beta-blockers are the potential systemic adverse effects that can result due to systemic absorption of the beta-blocker through the nasolacrimal duct. Some of these adverse effects include bradycardia, heart block, heart failure, and asthma. These adverse effects are especially concerning in patients with unknown underlying cardiac or pulmonary abnormalities.

In this study, the beta-blocker timolol maleate has been chosen for the treatment of patients with open-angle glaucoma due to its long-standing history of use and avoidance of cosmetic adverse effects. There are various drug delivery systems that can be used to administer ophthalmic timolol maleate. Timolol maleate solutions have many drawbacks due to their low viscosity, which results in a fast drug release rate from the solution. The ease of flow demonstrated by ophthalmic solutions allows for active timolol maleate to leave the site of action in the eye and drain into the systemic circulation.⁴ In the first seven years of commercial production of ophthalmic timolol maleate, there was an estimated 37 deaths caused by the product. It was theorized that these deaths resulted from the adverse cardiovascular, pulmonary, CNS, and endocrine effects that timolol maleate can cause when it is absorbed systemically.⁵ It is a challenge to properly identify patients at increased risk for timolol maleate adverse effects due to the vast array of patients with undiagnosed cardiovascular, pulmonary, CNS, and endocrine disorders.

Ophthalmic gels have been utilized to solve some of the problems experienced with the use of ophthalmic solutions. Ophthalmic gels can be preformed or in situ forming gels. An ideal in situ forming gel is a free-flowing liquid at non-physiologic conditions (pH 4.0 and 25° C.), but transitions into a strong gel with increased viscosity upon exposure to physiologic conditions (pH 7.4 and 37° C.). This transition allows consistent, reproducible administration of the drug via eye drops. In contrast, preformed gels are difficult to administer in a consistent, reproducible dose. Preformed ophthalmic gels are traditionally supplied in a tube and the patient is instructed to squeeze a ribbon of gel into the pocket of the lower eyelid; however dosing errors can easily occur. Ophthalmic gels can decrease the release rate of the drug, lengthen the residence time at the site of action, and decrease the drainage through the nasolacrimal duct. Less frequent dosing, utilization of lower drug concentrations, and decreased systemic absorption may all result from the use of gels compared to solutions. The main limitation of gels is the potential for discomfort and blurred vision due to the high viscosity of the gel.

Polymers are used to create ophthalmic gels. Carbomer is a high molecular weight non-linear polyacrylic acid, cross-linked polymer that possesses many beneficial properties. It is a non-newtonian polymer that exhibits pseudoplastic properties. Pseudoplastic properties are favorable for ophthalmic gels because increasing shear rates decrease their viscosity. The shear rate during blinking is approximately 10000 s⁻¹ and a decrease in viscosity at this shear rate will decrease resistance and improve comfort to the patient while blinking. Carbomer has a solution to gel transition as the pH is raised above 4.0. Physiologic pH is 7.4 indicating that carbomer will become a gel at the physiologic conditions of the eye. The solution form of the polymer, before administration, will allow for consistent dosing via an eye drop. In this study, we will be formulating a preformed gel; therefore, the benefit of in situ gelling properties will not be utilized. The difficulty in consistent dosing of an ophthalmic gel will be a barrier to the use of the products used in this experiment. Carbomer also has a high yield value of 5,500 dyn/cm², which increases its suspending ability. The clarity of carbomer, which is of particular interest in ophthalmic delivery systems, is high at concentrations less than 0.5%.⁸ Carbomer polymers have carboxyl functional groups incorporated into their chemical structures that facilitate an intrinsic buffering capacity. Although human tears have buffering capacity themselves, to avoid discomfort upon the administration of ophthalmic products, pharmacologic preparations should be as close to the pH of tears, 7.4, as possible. The buffering capacity of carbomer will assist in this. In previous studies comparing carbomer gels and ophthalmic solutions, carbomer gels have demonstrated decreased systemic absorption, flatter drug concentration versus time profiles, and lower effective drug concentrations. A good safety profile with the use of carbomer has also been established as it has been used in a variety of dosage forms, including ophthalmic, topical, oral, rectal, vaginal, and buccal products.

Carbomer polymers come in different grades. In this study carbomer 980 will be used. The viscosity of carbomer is dependent on the concentration of the polymer. Carbomer 940 and 980 have the highest viscosities at lower concentrations compared to the other carbomer grades. Since viscosity has been shown to increase the retention of ophthalmic drugs in the eye, a high viscosity would be desirable. Carbomer 940 did have a slightly increased viscosity over carbomer 980. Carbomer 940 and 980 have other similar properties including high clarity, low relative ion tolerance, and high relative shear tolerance. These are all desirable properties of an ophthalmic gel except for the low ion tolerance, which will be addressed in a later discussion. High clarity is a desirable property due to the administration of these products in the eye. High relative shear tolerance will allow for the polymer to remain intact while the polymer is being manipulated. Overall, carbomer 940 and 980 possess properties that would be beneficial when making an ophthalmic gel. 0.5% carbomer 980 will be used in the gels in this experiment. This polymer concentration was selected because below 0.2% a strong gel cannot form at physiologic conditions, but if the concentration is greater than 0.5% the gel becomes stiff, increasing the risk for patient discomfort, and acidic, making the gel difficult to neutralize. At concentrations greater than 0.5% the clarity also decreases.⁸

Solid calcium phosphate nanoparticles will also be added to the carbomer gel. The goal of the calcium phosphate nanoparticles will be to further slow down the release rate of timolol maleate. As previously mentioned, increasing the viscosity of a gel increases the retention time of the drug within the gel and at the site of action and decreases the systemic absorption, but it is at the expense of increased blurred vision and decreased comfort. The addition of the calcium phosphate nanoparticles will potentially have the effect of increasing the retention time of the drug without further increasing the viscosity of the gel. The retention time may be increased because the calcium phosphate nanoparticles will act as additional barriers the drug must get around in order to be released from the carbomer gel.

In regards to the incorporation of calcium phosphate nanoparticles, carbomer also has desirable properties such as the presence of carboxyl functional groups and a high yield value. The carboxyl functional groups can form strong bonds with the calcium phosphate nanoparticles aiding in dispersion and the high yield value will allow for better suspension of the timolol maleate and calcium phosphate nanoparticles. Potential disadvantages of carbomer with the use of calcium phosphate nanoparticles includes its low ion tolerance, but this is not expected to be a problem due to the insoluble nature of the calcium phosphate nanoparticles.

### Objectives

The purpose of this study is to determine the release rate of timolol maleate from carbomer gels with varying concentrations of calcium phosphate nanoparticles incorporated into the gel. If a slowed release rate of timolol maleate results due to the obstacles created by the calcium phosphate nanoparticles, the consequences of an increased retention time in the eye and decreased systemic absorption of timolol maleate may be observed. The discovery of an ophthalmic drug delivery system with these properties may lead to fewer adverse drug events, less frequent dosing, lower required drug concentrations, better tolerability, and better patient compliance of ophthalmic pharmacologic products.

### Methods

The overall design of this experiment is to determine the release rate of timolol maleate from ophthalmic carbomer gels that consist of timolol maleate and varying concentrations of calcium phosphate nanoparticles. This was accomplished by creating several timolol maleate preparations, including solutions, gels, and gels consisting of calcium phosphate nanoparticles. The preparations were inserted into dialysis tubing and the tubing was placed in beakers of phosphate-buffered saline. Due to the presence of sink conditions, the timolol maleate flowed from the inside of the dialysis tubing into the beakers. Samples were taken from the beaker and the concentration of timolol maleate was determined via absorbance measurements. The release rates of the preparations were compared to determine if the addition of calcium phosphate nanoparticles to the carbomer gel decreased the release rate of timolol maleate. The affect of the concentration of the calcium phosphate nanoparticles on the release rate of timolol maleate was also be explored.

The first step of this study was to create a standard calibration curve to determine the relationship between the absorbance and concentration of timolol maleate. Beer's Law was utilized to determine this relationship, with 10 cm equaling the length of path of radiation passing through due to the size of the cuvette. Timolol maleate salt and phosphate-buffered saline were combined to create timolol maleate solutions with the concentrations of 10%, 20%, 40%, 60%, 80%, and 100% of the total possible concentration of timolol maleate that would be present in the 100 mL beakers of phosphate-buffered saline during the release rate studies. The total possible concentration of timolol maleate was calculated by noting that 1 mL of 0.68% timolol maleate was placed in a dialysis tube that was placed in a 100 mL beaker of phosphate-buffered saline. The timolol maleate left the dialysis tubing and was diluted in the 100 mL of phosphate-buffered saline. Therefore, 1 mL of 0.68% timolol maleate in 101 mL of solution resulted in a total possible concentration of 0.06733 mg/mL. A stock solution of 0.0067 g/100 mL was made with phosphate-buffered saline. Of this stock solution, 1 mL, 2 mL, 4 mL, 6 mL, 8 mL, and 10 mL were combined with phosphate-buffered saline to make a total of 10 mL of solution with 10%, 20%, 40%, 60%, 80%, and 100% of the total concentration of timolol maleate, respectively. A UV-Visible Spectrophotometer was used to measure the absorbance of each solution. The varying concentrations of timolol maleate resulted in different amounts of absorbed radiation. A plot of absorbance versus concentration was constructed and a linear regression was used to generate the equation of the line. The equation of the line corresponds to Beer's Law, which allowed for the identification of a timolol maleate concentration given the absorbance of a solution or gel. If the absorbance of a timolol maleate solution or gel remains in the linear region of the absorbance versus concentration plot, absorbance and concentration can be used interchangeably due to their direct relationship. Due to this relationship, absorbance of timolol maleate versus time was plotted to demonstrate the release rate of timolol maleate from the various preparations.

Next, the dialysis tubing, cellulose ester dialysis membrane, was taken out of the refrigerator. The dialysis tubing with a molecular weight cut off (MWCO) of 3,500-5,000D was used. The dialysis tubing was rinsed with distilled water three times to remove the preservatives and then soaked in phosphate-buffered saline overnight. At this point, the dialysis tubing was ready to be filled with the timolol maleate solutions and gels.

The timolol maleate solutions were made by mixing 0.068 g of timolol maleate salt with 10 mL of phosphate-buffered saline to create a solution that is 0.68% timolol maleate. To ensure uniformity, the solution was inverted five times and a stir bar was inserted into the solution while it was placed on a magnetic stirrer. The pH and tonicity of the solution was not adjusted because the phosphate-buffered saline is isotonic and has a pH of 7.

A timolol maleate gel was constructed by first creating a concentrated formulation of carbomer 980 gel by adding distilled water to its solid formulation. The carbomer gel consisted of 0.5% carbomer 980; however, a 2% carbomer preparation was utilized to make the various gels. 125 mL of distilled water and 2.5 g of carbomer 980 were mixed by a stir bar and magnetic stirrer over night to create the 2% carbomer stock gel. The gel was also mixed by hand. The pH of the final gel was adjusted to a pH of 7.0-8.0; however, a carbomer gel that was 0.05% was created first to approximate the amount of sodium hydroxide that would be needed to appropriately adjust the 0.5% carbomer gel. To accomplish this, 2.5 g of 2% carbomer gel was weighed and q.s. to 100 mL with distilled water. Small increments of 0.1 N and 1 N sodium hydroxide were added to the 0.05% carbomer gel until the pH was 7.0-8.0, which was detected by an electrode. Next, 0.5% carbomer gel was made by placing 25 mL of 2% carbomer gel in a 250 mL beaker. Approximately 80 mL of water was added to the gel and then 0.68 g of timolol maleate was incorporated into the gel. The gel was mixed by hand until all lumps had dissipated. The pH of the 0.5% carbomer gel with 0.68% timolol maleate was pH adjusted by adding slightly less than ten times the amount of sodium hydroxide that was needed to pH adjust the 0.05% carbomer gel. The electrode was removed from the gel and rinsed with distilled water to ensure all of the ingredients remained in the gel. The gel was q.s to 100 g with distilled water.

The exact osmolality 0.5% carbomer 980 gel was not known, but it was calculated using the osmolality of 0.3% carbomer 934.¹⁹ The 0.68% timolol maleate contributes 31.4 mOsm and the carbomer contributes approximately 42 mOsm. To make the gel isotonic, 4.1 g of 5% dextrose would need to be added to obtain an isotonic gel with a tonicity of 300 mOsm. However, the results of this experiment will be compared to the results of a similar experiment using carboxymethylcellulose, CMC. CMC does not require tonicity adjustments; therefore, dextrose was not added to the carbomer gel to allow for a more direct comparison between the gels. The gel was thoroughly mixed by hand and then with a stir bar and magnetic stirrer to ensure uniformity.

Carbomer 980 gels with timolol maleate and calcium phosphate nanoparticles were made using the same previously discussed steps for the carbomer gel; however, 100 g of 2% carbomer and 2.72 g of timolol maleate were used to make 350 g of gel. This gel was pH adjusted using the previously discussed method. 87.5 g of this gel was weighed and calcium phosphate nanoparticles were added to the gel. To obtain gels with 0.5%, 1%, 2%, and 5% calcium phosphate nanoparticles, 0.5 g, 1 g, 2 g, and 5 g of the solid calcium phosphate nanoparticles were added, respectively. The gels were q.s to 100 g and mixed with a stir bar and magnetic stirrer. The gels were then placed in the Breville overhead, electric mixer for an additional 15 minutes to ensure uniformity.

There was 73.8 g of 0.5% carbomer gel with 0.68% timolol maleate remaining after five gels were made. The remaining gel was used to make a 1% carbomer gel with 1% calcium phosphate nanoparticles. 0.4215 g of carbomer 980 was added to the remaining gel. The gel was mixed by hand and the pH was adjusted with sodium hydroxide until the pH was 7.0-8.0. The gel was q.s to 84.3 g. The gel was then placed in the Brivelle mixer. The viscosity of the gels was determined using the Haake 550 viscotester. A curvefit analysis was performed on the viscosity data with Ostwald de Waele and Bingham models. The Ostwald de Waele model allowed for the flow consistency index, K, and flow behavior index, n, to be calculated. The Bingham model calculated the yield value. The Haake 550 viscotester was also used to investigate the thixotropy of the gels.

The release rates of timolol maleate from the solution and gels were then determined. The gels with calcium phosphate nanoparticles were placed in a homogenizer for two minutes to ensure the particles were uniformly suspended in the gel. The dialysis tubing was cut into 10 cm segments. The bottom of the dialysis tubing was clipped. 1 mL of timolol maleate solution or 1 gram of timolol maleate gel was inserted into the dialysis tubing. A volumetric pipette was used to transfer 1 mL of the solution into the dialysis tubing. The gel was too thick to utilize a volumetric pipette for this transfer; therefore, a 24 mL syringe was filled with the gel. The 24 mL syringe was used to fill a 3 mL syringe with the gel. A plastic catheter tip was placed on the 3 mL syringe, which allowed for 1 mL of the gel to be transferred to the dialysis tubing. It was evident that there were air bubbles in the gel that made it difficult to precisely transfer 1 g of gel. This barrier was accounted for by weighing the 3 mL syringe before and after the gel was transferred to calculate the exact weight that gel inserted into the dialysis tubing. The top of the dialysis tubing was also clipped and placed in a 100 mL beaker of phosphate-buffered saline. A stir bar was placed in the bottom of the beaker to ensure the concentration of timolol maleate was uniform throughout the beaker. The top clip was placed on the rim of the beaker and parafilm was placed over the top of the beaker to hold the clip in place and limit evaporation. The beaker was then weighed to ensure that evaporation would be detected if it occurred throughout the experiment. Due to the sink conditions of the beaker, the timolol maleate was released from the dialysis tubing and flowed into the beaker of phosphate-buffered saline. The absorbance of the released timolol maleate was measured using the UV-Visible Spectrophotometer. A pipette was used to collect the solution from the beaker and it was placed in a cuvette for absorbance analysis. After the absorbance reading was taken, the sample was placed back into the beaker. The first absorbance reading was taken between minutes 5 and 10 for the solution and gels to initially assess how quickly the timolol maleate is being released from the dialysis tubing. Depending on how quickly the absorbance of timolol maleate was changing, readings were taken every 5 minutes, 10 minutes, 20 minutes, or 30 minutes. The release rate of timolol maleate was measured for six samples of each formulation. As the experiment progressed, it became evident that the timolol maleate release rate of one of the 0.5% gel samples varied from the rest of the samples. Due to this, one sample was added to result in a total of seven samples for the 0.5% gel formulation.

The absorbance for the gels was then normalized to 1 g to allow for a uniform comparison between all of the samples. This was done by dividing 1 by the weight of gel in the sample. The resulting number was multiplied by the absorbance readings for the sample. The normalized absorbances were then converted to percentages of their maximum normalized absorbance. The percent of normalized absorbance was plotted versus time to represent the release rate of timolol maleate. The linear portion of each plot was isolated and a linear regression was used to determine the slope. The slope is a representation of how quickly the timolol maleate was released from the solution and gels. The average slope of the six samples of each formulation was calculated to compare the release rate of timolol maleate from the different preparations.

The data was analyzed using KaleidaGraph, a curve-fitting program. The non-linear regression was determined for the release rate of timolol maleate from the various preparations. The data was fit with a sigmoid curve fit model and the sigmoid parameters were expressed as m values. The m3 parameter correlated best to the timolol maleate release rate. An analysis of variance, ANOVA, statistical test was utilzed in this experiment because there were more than two groups to compare. It was able to determine if there was a statistical difference between the different timolol maleate preparations. A p-value of <0.05 indicated statistical significance. A post-hoc Tukey's Test was then used to determine which preparations were statistically different. ANOVA and a post-hoc Tukey's test were performed for the slopes and m3 values.

### Results

To further evaluate the release rates of timolol maleate from the solutions and gels, the slopes of the normalized percent absorbance versus time graph were calculated and are displayed in Table 1. The average slopes of the samples of each formulation concluded that the timolol maleate was released the slowest from the 1% gel with 1% calcium phosphate nanoparticles (CaP NP), closely followed by the 0.5% gel with 1% CaP NP. The 0.5% gel and 0.5% gel with 0.5% CaP NP were the next slowest, followed by the solution. Conversely, timolol maleate was released the quickest from the 0.5% gel with 5% CaP NP, followed by the 0.5% gel with 2% CaP NP. The slowed release rate of timolol maleate demonstrated by the preparations with 1% CaP NP was predicted; however, the hastened release rate of timolol maleate from the preparations with 2% and 5% CaP NP was unexpected. It was theorized that the addition of CaP NP would result in a slower release rate of timolol maleate, especially with high concentrations of CaP NP, but these result demonstrate that the slowed timolol maleate release rate is dependent on the concentration of CaP NP.

TABLE 1
Slopes of absorbance vs. time graph

| | | | 0.5% Gel | 0.5% Gel | 0.5% Gel | 0.5% Gel | 1% Gel |
|---|---|---|---|---|---|---|---|
| Sample | Solution | 0.5% Gel | 0.5% NP | 1% NP | 2% NP | 5% NP | 1% NP |
| 1 | 0.5248 | 0.4664 | 0.5289 | 0.3256 | 0.6186 | 0.8409 | 0.3586 |
| 2 | 0.5746 | 0.5481 | 0.4764 | 0.4893 | 0.5228 | 0.6898 | 0.3894 |
| 3 | 0.5818 | 0.3654 | 0.5245 | 0.3378 | 0.5683 | 0.6046 | 0.3629 |
| 4 | 0.4973 | 0.3782 | 0.4827 | 0.3850 | 0.6293 | 0.6459 | 0.3820 |
| 5 | 0.6417 | 0.7177 | 0.5369 | 0.4318 | 0.5982 | 0.5580 | 0.3959 |
| 6 | 0.5134 | 0.4958 | 0.4635 | 0.4004 | 0.5588 | 0.7169 | 0.3700 |
| 7 | | 0.5426 | | | | | |
| Ave. | 0.5556 | 0.5020 | 0.5022 | 0.3950 | 0.5827 | 0.6760 | 0.3765 |

The results of the ANOVA statistical test of the slopes of the normalized percent absorbance versus time graph are displayed in Table 2. This test shows that there is a statistically significant difference between some of the timolol maleate preparations. The F value is the ratio of the variability between the groups compared to the variability within the groups. A large F value indicates that the ratio is large and there should be a small p value indicating statistical significance. The ANOVA test revealed a large F value of 13.084832 and a small, statistically significant p-value of <0.0001. The post-hoc Tukey's test was then performed to determine which preparations differed in a statistically significant manner. The results of this test are located in Table 3. There were several preparations that differed. In general, the 0.5% gel with 5% Cap NP was significantly different from 1% gel with 1% CaP NP, 0.5% gel with 1% CaP NP, the 0.5% gel, and the 0.5% gel with 0.5% CaP NP due to its fast timolol maleate release rate. The 0.5% gel with 2% CaP NP was significantly different from the gels with 1% CaP NP, which was likely due to the increased timolol maleate release rate of the 0.5% gel with 2% CaP NP and decreased timolol maleate release rate of the gels with 1% CaP NP compared to the solution. The gels with 1% CaP NP were also significantly different from the solution due to this slowed timolol maleate release rate. Lastly, the 1% gel with 1% CaP NP, which had the slowest release rate of all the preparations, was significantly different from the 0.5% gel.

TABLE 2
ANOVA of Slopes

| Source | DF | SS | MS | F | P |
|---|---|---|---|---|---|
| Setting | 6 | 0.39641339 | 0.066068898 | 13.084832 | <.0001 |
| Error | 36 | 0.1817738 | 0.005049274 | | |
| Total | 42 | 0.57818724 | 0.013766363 | | |

TABLE 3
Post Hoc Tukey's Test of Slopes

| Formulation | Mean Difference | q | P | 95% CI |
|---|---|---|---|---|
| 0.5% Gel 5% CaP vs | 0.29955 | 10.326 | <.0001 | 0.17152 to 0.42758 |
| 1% Gel 1% CaP | | | | |
| 0.5% gel 5% CaP vs | 0.281033 | 9.6877 | <.0001 | 0.153 to 0.40907 |
| 0.5% gel 1% CaP | | | | |
| 0.5% gel 5% CaP vs | 0.173988 | 6.2241 | 0.0016 | 0.050611 to 0.29737 |
| 0.5% gel | | | | |
| 0.5% gel 5% CaP vs | 0.173867 | 5.9935 | 0.0026 | 0.045832 to 0.3019 |
| 0.5% gel 0.5% CaP | | | | |
| 0.5% gel 5% CaP vs | 0.120417 | 4.151 | 0.0767 | −0.0076177 to 0.24845 |
| solution | | | | |
| 0.5% gel 5% CaP vs | 0.09335 | 3.2179 | 0.2833 | −0.034684 to 0.22138 |
| 0.5% gel 2% CaP | | | | |
| 0.5% gel 2% CaP vs | 0.2062 | 7.108 | 0.0003 | 0.078166 to 0.33423 |
| 1% gel 1% CaP | | | | |
| 0.5% gel 2% CaP vs | 0.187683 | 6.4697 | 0.001 | 0.059649 to 0.31572 |
| 0.5% gel 1% CaP | | | | |
| 0.5% gel 2% CaP vs | 0.0806381 | 2.8847 | 0.4087 | −0.042739 to 0.20402 |
| 0.5% gel | | | | |
| 0.5% gel 2% CaP vs. | 0.0805167 | 2.7755 | 0.4547 | −0.047518 to 0.20855 |
| 0.5% gel 0.5% CaP | | | | |
| 0.5% gel 2% CaP vs | 0.0270667 | 0.933 | 0.994 | −0.10097 to 0.1551 |
| solution | | | | |
| Solution vs | 0.179133 | 6.175 | 0.0018 | 0.051099 to 0.30717 |
| 1% gel 1% CaP | | | | |
| Solution vs | 0.160617 | 5.5367 | 0.0065 | 0.032582 to 0.28865 |
| 0.5% gel 1% CaP | | | | |
| Solution vs | 0.0535714 | 1.9164 | 0.8211 | −0.069806 to 0.17695 |
| 0.5% gel | | | | |
| Solution vs | 0.05345 | 1.8425 | 0.8461 | −0.074584 to 0.18148 |
| 0.5% gel 0.5% CaP | | | | |
| 0.5% gel 0.5% CaP vs | 0.125683 | 4.3325 | 0.0572 | −0.002351 to 0.25372 |
| 1% gel 1% CaP | | | | |
| 0.5% gel 0.5% CaP vs | 0.107167 | 3.6942 | 0.1524 | −0.020868 to 0.2352 |
| 0.5% gel 1% CaP | | | | |
| 0.5% gel 0.5% CaP vs | 0.000121437 | 0.0043 | 1 | −0.12326 to 0.1235 |
| 0.5% gel | | | | |
| 0.5% gel vs | 0.125562 | 4.4917 | 0.0438 | 0.0021849 to 0.24894 |
| 1% gel 1% CaP | | | | |
| 0.5% gel vs | 0.107045 | 3.8293 | 0.1255 | −0.016332 to 0.23042 |
| 0.5% gel 1% CaP | | | | |
| 0.5% gel 1% CaP vs | 0.0185167 | 0.6383 | 0.9993 | −0.10952 to 0.14655 |
| 1% gel 1% CaP | | | | |

Bold indicates statistical significance

The normalized percent absorbance versus time graphs representing the timolol maleate release rates for the solution and gels were analyzed using the KaleidaGraph program. A curvefit analysis was performed and it was determined that a sigmoid model best fit the data. The sigmoid curve is represented by the values m1, m2, m3, and m4. The average of the m-values for each formulation were calculated and are displayed in Table 4. The m3 values most closely relate to the timolol maleate release rate. A high m3 value indicates a slow release rate. The 1% gel with 1% CaP NP had the highest m3 value followed by the 0.5% gel with 1% CaP NP. The lowest m3 value was for the 0.5% gel with 5% CaP NP, followed by the 0.5% gel with 2% CaP NP. The average m-values were used to create a sigmoid curve that allows the timolol maleate release rates to be compared between the different preparations.

TABLE 4
Average m-values from sigmoid curve fit model

| | | | 0.5% Gel | | | | 1% Gel |
|---|---|---|---|---|---|---|---|
| | | | 0.5% CaP | 0.5% Gel | 0.5% Gel | 0.5% Gel | 1% CaP |
| | Solution | 0.5% Gel | NP | 1% CaP NP | 2% CaP NP | 5% CaP NP | NP |
| m1 | 114.4783 | 105.2558 | 103.8783 | 102.3307 | 103.9817 | 104.03 | 100.1408 |
| m2 | 2.379183 | 1.891809 | 0.39668 | 0.769753 | 1.201202 | 1.274197 | −0.93045 |
| m3 | 116.6945 | 93.69814 | 115.5217 | 140.5237 | 92.70183 | 80.97017 | 155.2583 |
| m4 | 1.346883 | 1.435571 | 1.671917 | 1.467833 | 1.659617 | 1.684783 | 1.416817 |

The ANOVA statistical test revealed that there were some statistically significant differences between the m3 values and the post-hoc Tukey's test highlighted which preparations were statistically different from each other. The results from the ANOVA and from the post-hoc Tukey's test are in Table 5 and Table 6, respectively. The results for the slopes and the m3 values both reflect the timolol release rates. The trends of the slopes and m3 values are closely, but not exactly, correlated. The linear trendline has an r² value of 0.8119, which is statistically significant. Both the slopes and m3 values as representations of the release rate produced useful data and their strong correlation strengthen overall release rate results.

TABLE 5
ANOVA of m3 values

| Source | DF | SS | MS | F | P |
|---|---|---|---|---|---|
| Setting | 6 | 26614.339 | 4435.7231 | 12.282842 | <.0001 |
| Error | 36 | 0.18177385 | 0.005049274 | | |
| Total | 42 | 39615.078 | 943.21615 | | |

TABLE 6
Post-Hoc Tukey's Test for m3 values

| | | | | |
|---|---|---|---|---|
| | Mean | | | |
| Formulation | Difference | q | p | 95% CI |

| | | |
|---|---|---|
| 1% gel 1% CaP NP vs 0.5% gel 5% | <.0001 | 40.047 to 108.53 |
| CaP NP | | |
| 1% gel 1% CaP NP vs 05% gel 2% | <.0001 | 28.316 to 96.797 |
| CaP NP | | |
| 1% gel 1% CaP NP vs 0.5% gel | <.0001 | 28.565 to 94.556 |
| 1% gel 1% CaP NP vs 0.5% gel 0.5% | 0.0142 | 5.4958 to 73.978 |
| CaP NP | | |
| 1% gel 1% CaP NP vs solution | 0.0188 | 4.323 to 72.805 |
| 1% gel 1% CaP NP vs 0.5% gel 1% | 0.8271 | −19.506 to 48.976 |
| CaP NP | | |
| 0.5% gel 1% CaP NP vs 0.5% gel 5% | <.0001 | 25.313 to 93.794 |
| CaP NP | | |
| 0.5% gel 1% CaP NP vs 0.5% gel 2% | 0.0019 | 13.581 to 82.063 |
| CaP NP | | |
| 0.5% gel 1% CaP NP vs 0.5% gel | 0.0015 | 13.83 to 79.821 |
| 0.5% gel 1% CaP NP vs 0.5% gel | 0.2817 | −9.2389 to 59.243 |
| 0.5% CaP NP | | |
| 0.5% gel 1% CaP NP vs solution | 0.3353 | −10.412 to 58.07 |
| Solution vs 0.5% gel 5% CaP NP | 0.0361 | 1.4835 to 69.965 |
| Solution vs 0.5% gel 2% CaP NP | 0.3275 | −10.248 to 58.234 |
| Solution vs 0.5% gel | 0.3336 | −9.999 to 55.992 |
| Solution vs 0.5% gel 0.5% CaP NP | 1 | −33.068 to 35.414 |
| 0.5% gel 0.5% CaP NP vs 0.5% gel | 0.0467 | 0.31063 to 68.792 |
| 5% CaP NP | | |
| 0.5% gel 0.5% CaP NP vs 0.5% 2% | 0.3856 | −11.421 to 57.061 |
| CaP NP | | |
| 0.5% gel 0.5% CaP NP vs 0.5% gel | 0.3946 | −11.172 to 54.819 |
| 0.5% gel vs 0.5% vs gel 5% CaP NP | 0.8881 | −20.267 to 45.723 |
| 0.5% gel vs 0.5% vs gel 2% CaP NP | 1 | −31.999 to 33.992 |
| 0.5% gel 2% CaP NP vs 0.5% gel 5% | 0.933 | −22.509 to 45.973 |
| CaP NP | | |

The viscosity was measured for each gel. The 1% gel with 1% CaP NP had the highest viscosity followed by the 0.5% gel with 5% CaP NP, the 0.5% gel with 2% CaP NP, the 0.5% gel with 0.5% CaP NP, the 0.5% gel, and finally the 0.5% gel with 1% CaP NP, which had the lowest viscosity. The viscosity results did not correlate with the timolol maleate release rates.

The viscosity curves were fitted by the Ostwald de Waele and Bingham models and the results are displayed in Table 7. The thixotropy was also evaluated for each gel and the results are in Table 7. The 0.5% gel with varying concentrations of CaP NP displayed a linear relationship for the K and the yield value. Due to the significant difference in viscosity of the 1% gel compared to the 0.5% gels, the 1% gel was not included in the comparison of rheology properties. The smallest K value was produced by the 0.5% gel with 1% CaP NP followed by the 0.5% gel, the 0.5% gel with 0.5% CaP NP, the 0.5% gel with 5% CaP NP, and lastly the 0.5% gel with 2% CaP NP. The linear relationship between these gels had an r² value of 0.87246, which corresponds to a statistically significant p-value of <0.01. The yield value results had a similar trend with the 0.5% gel with 1% CaP NP resulting in the lowest yield value followed by the 0.5% gel, the 0.5% gel with 0.5% CaP NP, the 0.5% gel with 2% CaP NP, and then the 0.5% gel with the 5% CaP NP. The r² value of the yield value relationship was 0.92558, which indicates a statistically significant p-value of <0.001. The trends between these values were slightly different in regards to the order of the 0.5% gels with 2% and 5% CaP NP; however, the K and yield values were very similar for these gels. The previously discussed release rate results demonstrated a similar trend for the 0.5% gels with the slowest release rate produced by the 0.5% gel with 1% CaP NP and the fastest release rates produced by the 0.5% gel with 5% CaP NP, closely followed by the 0.5% gel with 2% CaP NP. Therefore, there was a correlation between the release rates and the gels and their corresponding K values and yield values. The m3 values produced a similar trend; however the smallest m3 value correlated with the largest values for the K and yield values. This relationship represents the same relationship as the average slopes because the smallest m3 value indicated the fastest release rate. The relationship between the n and the thixotropy results did not produce a linear relationship between the 0.5% gels. The n values were all less than 1. An n value less than 1 indicates that the gel is exhibiting pseudoplastic behavior, which was an expected finding. The thixotropy results produced negative values. A negative thixotropy results indicate that the gel had a lower viscosity when the shear rate increased from 0 1/s to 1000 1/s compared to when the shear rate was decreased from 1000 1/s to 0 1/s. The 0.5% gel with 5% calcium phosphate nanoparticles had a thixotropy result that was much more negative than the rest of the gels.

TABLE 7
Rheology Properties

| | | 0.5% Gel | 0.5% Gel | 0.5% Gel | 0.5% Gel | 1% Gel 1% |
|---|---|---|---|---|---|---|
| | 0.5% Gel | 0.5% CaP NP | 1% CaP NP | 2% CaP NP | 5% CaP NP | CaP NP |
| K | 6.319 | 7.004 | 5.022 | 9.709 | 9.688 | 55.55 |
| n | 0.4293 | 0.4179 | 0.4416 | 0.4021 | 0.4154 | 0.3298 |
| Thixotropy | −5157.5 | −2633 | −4412 | −4903 | −10617.5 | −1384 |
| Yield | 43.59 | 46.40 | 36.71 | 59.92 | 63.06 | 249.90 |
| Value | | | | | | |

### Discussion

The timolol maleate release rates for the solutions and gels highlighted by the slopes of the normalized percent absorbance versus time in Table 1 presented unexpected results. It was theorized that the addition of calcium phosphate nanoparticles to a carbomer gel would slow down the release rate of timolol maleate; however, gels of this type had not been studied. The timolol maleate release was expected to progressively slow down as more calcium phosphate nanoparticles were added to the carbomer gel; however, this was not seen. As expected, the release rate was slower for the 0.5% carbomer gel compared to the solution. When 1% calcium phosphate nanoparticles were added to the 0.5% carbomer gel, the release rate was also decreased. However, when 5% calcium phosphate nanoparticles were added to the 0.5% carbomer gel, the timolol maleate release rate was increased, which was not anticipated. This phenomenon was further investigated by measuring the timolol maleate release rate of a 0.5% gel with 0.5% and 2% calcium phosphate nanoparticles. The addition of 0.5% calcium phosphate nanoparticles slightly increased the release rate of timolol maleate compared to the 0.5% gel, but the release rate was very similar to the 0.5% gel. This possibly indicated that 0.5% calcium phosphate nanoparticles is not a high enough concentration to make a significant difference in the timolol maleate release rate. The addition of 2% calcium phosphate nanoparticles resulted in a timolol maleate release rate that was faster than the solution. Therefore, the 0.5% carbomer gel with 2% calcium phosphate nanoparticles, similar to the 0.5% carbomer gel with 5% calcium phosphate nanoparticles, was not effective at decreasing the release rate of timolol maleate. Since the addition of 1% calcium phosphate nanoparticles to the 0.5% carbomer gel was the most effective in decreasing the release rate of timolol maleate, a 1% carbomer gel with 1% calcium phosphate nanoparticles was investigated to determine if the calcium phosphate nanoparticles would remain effective in decreasing the release rate. The timolol maleate release rate from the 1% carbomer gel with 1% calcium phosphate nanoparticles had a similar release rate to the 0.5% carbomer gel with 1% calcium phosphate nanoparticles, but the timolol maleate release rate was slightly slower. The results from the ANOVA statistical test and post-hoc Tukey's test revealed that there were some statistically significant differences between the timolol maleate preparations; however, some preparations were too similar to produce statistically different slopes. Overall, it was evident that only certain concentration of the calcium phosphate nanoparticles produced a slower timolol maleate release rate. The slowed timolol maleate release rate appeared to be further decreased as the concentration of the polymer was increased, but more studies are needed to confirm this effect since calcium phosphate nanoparticles in carbomer gel have not previously been studied. It appears that there could be an interaction between the carbomer gel and calcium phosphate nanoparticles contributing to the timolol maleate release rate results.

The m-values and sigmoid curves of the timolol maleate release rates also demonstrate that the different preparations produce different curves that correlate with the speed of the timolol maleate release rate. Due to the interpretation that the m3 parameter of the sigmoid equation is a representation of the timolol maleate release rate, an objective measurement of the m3 was able to reinforce the release rate determined by the average slope values. The m3 values did not exactly correlate with the average slopes, but they supported the data that revealed the 0.5% gel with 1% CaP NP and 1% gel with 1% CaP NP had the slowest release rates. The m3 values also reinforced that the 0.5% gel with 5% CaP NP and 0.5% gel with 2% CaP NP had the fastest release rates. This data suggests that the addition of CaP NP is able to slow the timolol maleate release rate, but only at certain concentrations. The m1, m2, and m4 parameters of the sigmoid curve fit model were also investigated. It appears that the m1 parameter is related to the height of the release rate curve and possibly the shape of the later curve portion of the graph. The m2 parameter appears to be related to lag time and the m4 parameter seems to be related to the overall shape of the curve. Since this experiment was most focused on the timolol maleate release rates, the m3 values were investigated the most.

The viscosity results did not correlate with the order of the timolol maleate release rates. In previous studies, the release rate of gels decreases as the viscosity increases. Therefore, the gels with fastest release rates would be expected to have the lowest viscosities and the gels with the slowest release rates would be expected to have the highest viscosities, but this was not seen in this study. The 1% gel with 1% calcium phosphate nanoparticles, which had the slowest release rate, did have the highest viscosity; however, this gel cannot be compared to the 0.5% gels. The unexpected viscosity results of the 0.5% gels reinforces the theory that there could be an interaction between the carbomer gel and calcium phosphate nanoparticles that does not contribute to a higher viscosity, but does produce a slower release rate. If this is reproduced in future studies, this could be a beneficial finding because increased viscosity often causes discomfort for patients using ophthalmic gels and it would be desirable to decrease the release rate of a drug without increasing the viscosity of the gel.

The trend of K and yield value for the 0.5% gels did generally follow the trend of increasing K and yield value with increasing release rate. At this point, the significance of these results is not known. The n value results of less than 1 for all gels suggest that the addition of CaP NP does not disturb the pseudoplastic properties of the carbomer gel. This is a promising finding because, as previously stated, an n value less than 1 indicates that the gel is exhibiting psuedoplastic behavior. Pseudoplastic behavior allows for gels to become less viscous as shear rates are increased. This is a desirable property for ophthalmic products because patient comfort can be maintained as the shear rate is increased due to blinking. The thixotropy results, highlighting the difference between the viscosity values as the shear rate is increasing and the viscosity as the shear rate is decreasing, did not demonstrate a clear trend. The thixotropy results were all negative, which is not common with many polymers; however, negative thixotropy was also seen with carbomer gels in a study by Malah. The thixotropy studies were also conducted twice to confirm the negative results. The only thixotropy value that was significantly different out of the gels was the 0.5% gel with 5% calcium phosphate nanoparticles. Since this was the gel with the highest concentration of calcium phosphate, and fastest timolol maleate release rate, there is a potential correlation between an increased thixotropy value and increased calcium phosphate nanoparticles. This correlation is not yet confirmed since it was only seen with one data point and more data is needed to confirm the presence of this result.

### Conclusion

The addition of 1% calcium phosphate nanoparticles to a carbomer gel was successful at slowing the timolol maleate release rate. A slowed timolol maleate release rate was not seen with carbomer gels with 2% and 5% calcium phosphate nanoparticles. At this point, it is thought that there could be an interaction between the carbomer gel and the calcium phosphate nanoparticles that is contributing to the slowed timolol maleate release rate at certain concentrations of calcium phosphate nanoparticles. Ophthalmic gels may be able to utilize calcium phosphate nanoparticles to decrease the release rate of the drug without further increasing the viscosity of the gel. Future studies are needed to confirm the effects of the calcium phosphate nanoparticles on carbomer gels.

### Example 2

An ophthalmic gel exhibits multiple components that determine its properties such as the polymer it is composed of. Carboxymethylcellulose (CMC), is a water-soluble, bioadhesive polymer that displays mucoadhesive properties and is biodegradable. The bioadhesive property allows for attachment of a drug to the polymer which permits an extended contact time of the drug to the ocular tissue and improved ocular bioavailability. Safety data for CMC per manufacturer show that there is no toxicity from animal and human studies. This lack of toxicity is what the manufacturers contribute to the frequent use and incorporation of this polymer in a multitude of products. The common adverse effects reported with Na CMC when used in lubricant eye drops are gritty/sandy eyes, blurry vision, dry eyes, decreased visual acuity, and eyelid margin crusting. CMC has film-forming properties which allow it to interact with tear film and this results in an increases in the polymer's stability. ⁵

Cellulose polymers and low molecular weight CMC exhibit newtonian properties which displays viscosity independent of shear rate and increase in viscosity which causes pain when blinking. However, due to the larger molecular weight of the Na CMC used in this study, non-newtonian and pseudoplastic properties are seen which leads to a decrease in viscosity with increasing shear rate. These properties permit an improved patient tolerability profile with less resistance when blinking. Gelation occurs with CMC with the addition of trivalent metals such as aluminum through a mechanism of cross-linking of the polymer molecules between the carboxy methyl groups. Gradual release of aluminum from monobasic aluminum acetate, soluble salts, or insoluble salts such as dihydroxyaluminum sodium carbonate causes in situ formation of the CMC.⁹ However, preformed gels will be implemented in this study which does not necessitate the incorporation of the gelation described above. The yield value for less viscous CMC is 36 dyn/cm², which is a value that indicates the initial resistance to flow under stress and is used to determine suspending ability.

CMC polymers can be made with varying degrees of viscosities based on molecular weight and temperature. Although not a factor in this study, CMC polymer viscosity is affected by temperature but only when it is for a long duration of heating at high temperatures (82° C. for >48 hours) and can reduce viscosity by 60%. The different types manufactured are low viscosity, moderate viscosity, and high viscosity. The higher viscosity CMC polymers have the highest molecular weight of 700,000 and degree of polymerization of 3200 compared to the lowest viscosity CMC polymers are molecular weight of 90,000 and degree of polymerization of 400. Degree of polymerization refers to the average length of chain and is consistent with the concept that the longer the chain, the more viscous the polymer is. The degree of substitution (DS) varies with each CMC polymer which further classifies the different types of CMC, for instance, DS 0.7 is the most frequently utilized and is adequate for ophthalmic drug delivery. DS 0.7, written as 7, allows for high viscosity due to the lower degree of substitution of the polymer compared to a DS 1.2, written as 12.

The highest viscosity of the different CMC polymers is indicated by an H, and in this study for Na CMC, 7HF PH will be utilized. As mentioned above, there are many different uses for CMC. The different types are distinguished with the letters, such as F for food or cosmetic, P for pharmaceuticals, PH for cosmetic or pharmaceutical, or none for industrial purposes. In this study, F PH is the grade used which is for the intended use of food, cosmetic, or pharmaceutical. The concentration of Na CMC used in this study was 1% and 3% which are the high viscosity types and will allow for the most ocular retention. Na CMC 1% is frequently used when the polymer has been studied in previous clinical trials as a lubricant eye drop. Na CMC 3% is the higher viscosity concentration that has clinically been proven to display gel-forming capabilities. Both Na CMC 1% and 3% have been verified as tolerable for the human eye, while allowing for the most viscosity attainable from this polymer. Increased viscosity is ideal for delaying drug release, however, too much viscosity can cause ocular discomfort and clarity could be compromised. This research will help differentiate the magnitude of variation between the two polymer concentrations. Na CMC is also advantageous as a polymer because it has the buffering capacity to regulate pH adjustments as necessary.

Kyyronen et al. studied timolol maleate with the addition of Na CMC and its result on systemic absorption. The results concluded that Na CMC with timolol maleate improved ocular concentrations by 3 to 9 fold compared to non-viscous eye drops. Also, this study showed that there was a reduction in the rate of systemic absorption of timolol maleate by 33% which mitigated the CV side effects seen in the other study group with no polymer inclusion. These benefits were attributed to the mucoadhesive effects of Na CMC allowing for longer corneal contact and slower spreading of the solution to the nasolacrimal gland. Jarvinen et al. compared CMC to carbopol in the systemic absorption of ophthalmic timolol maleate in rabbits. The two bioadhesive polymers showed that in equiviscous solutions, there was a similar decrease in systemic absorption and 50% decrease in AUC of timolol maleate in the plasma. These studies highlight the efficacy of Na CMC in preventing the systemic absorption of beta blocker agents which diminishes the occurrence of systemic CV complications.

As previously mentioned, increased viscosity does have a negative implication of ocular discomfort and blurry vision. To obtain the benefits of increased viscosity and prevent absorption systemically without the increased discomfort, the addition of calcium phosphate nanoparticles will be implemented and looked at in this study. This addition is a novel approach which will augment the polymer by causing the drug delivery to be further slowed down. The nanoparticles of calcium phosphate nanoparticles will allow for an increase in surface area with the polymer to block the pathway for drug delivery. Incorporation of nanoparticles in ophthalmic drug delivery could possibly require less polymer use while impeding the drug release rate and still keep active ingredient retained in the eye.

### Methodology

The study design for this experiment is the addition of calcium phosphate nanoparticles to timolol maleate and Na CMC polymer and its effect on the release rate of the drug. Varying concentrations of calcium phosphate nanoparticles (CaP NP) were used to distinguish if the amount of the nanoparticles correlated with further delay in drug delivery. Also, two different concentrations of Na CMC polymer were studied to assess whether the degree of viscosity had an effect on the release rate. This study incorporated different preparations of timolol maleate such as ophthalmic solution, ophthalmic gel, and ophthalmic gel with varying concentrations of calcium phosphate nanoparticles. A standard calibration curve for timolol maleate was developed initially for reference later in the study to determine the concentration of timolol maleate based on the absorbance values. The different preparations were placed in dialysis tubings which were then placed in a 100m1 beaker of phosphate-buffered saline. Timolol maleate flowed from the dialysis tubing to the beaker at different concentrations depending on the preparation. These values were plotted to determine if the release rate of timolol maleate was delayed with the addition of calcium phosphate nanoparticles and Na CMC polymer.

A standard calibration curve was developed through the use timolol maleate salt with phosphate-buffered saline, as described in Example 1.

There were 6 gels made for this study, summarized in Table 8

TABLE 8
CMC Gel formulations

| | Timolol | | |
|---|---|---|---|
| | Maleate (%) | Na CMC (%) | CaP NP (%) |
| 1% CMC gel | 0.68 | 1 | — |
| 3% CMC gel | 0.68 | 3 | — |
| 1% CMC, 1% CaP NP | 0.68 | 1 | 1 |
| gel | | | |
| 1% CMC, 5% CaP NP | 0.68 | 1 | 5 |
| gel | | | |
| 3% CMC, 1% CaP NP | 0.68 | 3 | 1 |
| gel | | | |
| 3% CMC, 5% CaP NP | 0.68 | 3 | 5 |
| gel | | | |

The desired concentrations of Na CMC polymer were 1% and 3% and timolol maleate was 0.68%. In order to reach these target concentrations, a more concentrated amount of polymer was used to create the gel, resulting in Na CMC polymer of 5% and 9%. First, the timolol maleate with 1% Na CMC and timolol maleate with 3% Na CMC gels were made. In order to make the concentrated polymer of 5%, a stock solution was made to make enough for 3 different gels of Na CMC. 5 g of the polymer was soaked with distilled water which was left overnight to allow for hydration and uniformity. Then, 33.3 ml of Na CMC and 0.68 g of timolol maleate salt were added together to make the 1% Na CMC. This gel was then q.s. to 100 ml with distilled water.

Also, to make the concentrated 9% of the polymer to create enough for 3 gels, 9 g of the polymer was added to a 250 ml beaker. The polymer was hydrated with distilled water to 180 ml which took 1 hour to dissolve through the use of magnetic stirrer and stirring by hand. Next, 0.68 g of timolol maleate salt was added to the beaker with 60 ml of Na CMC. The contents were then q.s to a final concentration of 100 ml. The beaker was mixed with spatula to create a uniform dispersion of the drug with the polymer. It was important to ensure the product was isotonic to 300 mOsm which allowed for the results to be extrapolated for clinical use for glaucoma without causing eye irritation. Timolol maleate has osmolality of 31 mOsm and Na CMC has a theoretical osmolality of 470 mOsm which mitigated the need to add dextrose to reach osmolality.¹⁸ The pH was assessed by using an electrode and the pH was between 7-8 which prevented the need to add sodium hydroxide to get to the physiological pH. The mixture was placed in the Breville Scraper Mixer to further create uniformity of the mixture for an additional 10 minutes.

The same approach was implemented for the remainder of the gels formed with the addition of the varying concentrations of calcium phosphate nanoparticles. In order to create 1% and 5% strengths of calcium phosphate nanoparticles, it required incorporating 1 g and 5 g of the nanoparticles, respectively. For the 1% of Na CMC, 33.3 ml of the polymer was added to 0.68 g of timolol maleate and for the 3% of Na CMC, 60 ml of the polymer was added to 0.68 g of timolol maleate. Then, either 1 g or 5 g was added of calcium phosphate nanoparticles to reach the 1% and 5% for each preparation, respectively. The contents were q.s. to a final concentration of 100 ml and were mixed with a spatula to create a uniform dispersion of the drug with the polymer. Finally, the contents were placed in the Breville Scraper Mixer which created further uniformity of the mixture for an additional 15 minutes. All of the gel formulations were homogenous and uniform through use of homogenizer for 2 minutes at speed 24 before starting the release rate studies. Moreover, the gel viscosities were assessed using the Haake Viscotester 550. The viscosities studies were duplicated to confirm the varying degrees of viscosities were reproducible and precise. Curve-fit analysis was performed using the Bingham and Ostwald-De Waele model. The Bingham model allowed for the calculation of the yield value for each gel preparation. The Ostwald-De Waele model allowed for the flow consistency index (k) and flow behavior index (n) to be determined. Also, thixotropy analysis was assessed to determine the shear thinning properties of each preparation through the use of the Haake Viscotester 550.

The 10 cm precut dialysis tubings previously discussed were filled with either 1 g of gel or 1 ml of solution. Each formulation of gel and the solution had at least 6 dialysis tubings to account for error and reproducibility. Once the solution or gel was placed in the dialysis tubing, the top of the tubing was clipped and timer was started. The first release rate study conducted was of the timolol maleate solution. The solution was placed in dialysis tubing through the use of 1 ml volumetric pipette. Next, the dialysis tubing with the solution inside was placed in a beaker with 100 ml of phosphate-buffered saline. A stir bar was placed in the beaker and beakers were left on magnetic stirrers to keep the preparation uniform. The beaker was enclosed with a parafilm cover to prevent evaporation and loss of the solution. The weight of the beaker was documented for each sample to assess if evaporation occurred. A sample concentration from the dialysis tubing was obtained through a pipette and placed in cuvette to assess timolol maleate released from the dialysis tubing to the beaker. These samples were then placed into the UV visible spectrophotometer to distinguish the absorbance measurements. The time was documented when each sample was assessed throughout the release rate study. These absorbance measurements were used to determine the concentration of timolol maleate as previously discussed. For the release rate study of the gel formulations, a different method was used to draw up the gels to put in the dialysis tubings due to the increased viscosity of the preparations. The different preparations of gel were placed in a 24 ml syringe and then a 3 ml syringe was used to draw up 1 ml of the gel from the 24 ml syringe. The 3 ml syringe was weighed before placing the gel in the tubing and was reweighed after the 1 ml was placed in dialysis tubing. The difference of the two weights determined the amount of gel in the tubing. This method allowed for an accurate depiction of the amount of gel placed in the dialysis tubings and allowed for normalization of the data to factor in air bubbles that were present in the syringe. The rest of the procedure for the gel release rate studies was the same as the solution as described above.

The frequency at which samples were checked was conducted was based on if the preparation was a solution or gel and if calcium phosphate nanoparticles were added. For example, the interval of checking ophthalmic solution was shorter with the first checking at 5 minutes, 10 minutes, and 15 minutes. The gel formulations (without the calcium phosphate nanoparticles) were checked at 5 minutes and then every 10 minutes. As the absorbance values were starting to slow down, the time interval was increased to every 20 minutes and then every 30 minutes. The amount of timolol maleate released in the ophthalmic gel at those earlier times helped to determine how often to check the remainder of the duration. For instance, when the timolol maleate release was minimal at 10 minutes, then the next interval was extended for a longer duration at 25 minutes rather than 10 minutes. It was expected that the gel formulations with the calcium phosphate particles would have the longest delay in timolol maleate release. However, the first initial sample was still obtained at 5 minutes and then based on the level, further intervals were determined at that point. The beakers were weighed after 24 hours to ensure there was no loss of preparation. The weight of the beaker was compared to the weight noted at the beginning of the release rate study. The difference in weight was attributed to evaporation and distilled water was added to reach the weight as the beginning of the study and one last absorbance reading was taken.

As previously mentioned, each of the different preparations had at least 6 different tubings allowing for 6 different samples to be tested. This allowed for more accurate data, reproducibility, and less error attributed to chance. As the release rate studies progressed for the 1% CMC gel and the 1% CMC with 1% calcium phosphate nanoparticles (CaP NP) gel, there was some variation of two of the samples from the remainder of the samples. This led to the addition of two more samples per preparation; therefore 8 samples were tested. The 3% CMC with 5% CaP NP gel had one sample which was not properly clipped and placed in the phosphate-buffered saline. This sample displayed absorbencies that were not consistent with the other 5 samples and was considered an outlier. These results were then plotted in an absorbance versus time graph. The linear portion of the release rate results allowed for extrapolating to a concentration of timolol maleate versus time graph. The data for the gel formulations was normalized based on the amount of gel that was included in the dialysis tubing. The data was normalized by dividing the amount of gel intended per tubing (1 g) over the actual amount present and that was the factor used to multiply each absorbance for that sample. This allowed for all the samples to be directly compared to each other in that preparation. Also, the normalized absorbencies for the gels and the absorbance for the solution were assessed based on percent absorbance. In order to obtain percent absorbance, each normalized absorbance (for gels) or absorbance (for solution) were divided by the highest absorbance for that sample which was determined to be maximum release of timolol maleate. The highest absorbance of the sample was divided by 100 to get the percent absorbance. This allowed for each sample to be evaluated to each other in that preparation as well as all the other preparations.

The statistical analysis was performed using the KaleidaGraph Version 4 program and a curve fitting program was used that best fit the release rate data to determine the nonlinear regression data. ANOVA test was used to analyze the difference between the preparation groups. ANOVA determined the variance of all the different preparations and determined how strong the correlation was between the different variances. A p-value of <0.05 was considered statistically significant. A post-hoc test, Tukey's test was performed to allow for multiple comparisons of the release rate studies to determine which data is statistically different from the various preparations.

### Results

Each sample's percent absorbance versus time was plotted, showing that the different preparations resulted in different release rates. This graph was further investigated by taking the linear portion of each sample to determine the slope of each line to directly compare each other. The average slopes of each preparation highlighted that the solution had the highest release rate which was expected (table 9). The 1% CMC gel did not significantly delay the release rate of timolol maleate compared to the solution. However, the addition of 1% calcium phosphate nanoparticles greatly delayed release rate of the active drug compared to 1% CMC gel. It was expected that the 5% calcium phosphate nanoparticles would have further delayed release; however, the opposite was seen with the release rate was seen as higher than the 1% calcium phosphate nanoparticles. The 3% CMC gel proved to delay the release rate due to increased viscosity compared to all the 1% CMC formulations. Consistent with the 1% CMC with 1% CaP NP gel, the 3% CMC with 1% calcium phosphate nanoparticles gel was also slower than the gel by itself. However, once again, the addition of 5% calcium phosphate nanoparticles increased the release rate compared to the 1% calcium phosphate nanoparticles. This indicates that although calcium phosphate nanoparticles does delay release of the timolol maleate compared to just polymer with drug alone, there is a specific concentration of the nanoparticle that should be used. There is not a concentration dependent correlation with the higher the calcium phosphate nanoparticles, the longer the delay in release rate. Also, the 1% CMC gel compared to the 3% CMC gel was much faster indicating the viscosity of the polymer did have a correlation with release rate. The addition of a viscous polymer of 3% of Na CMC with 1% calcium phosphate nanoparticles proved to provide the slowest release rate of all 7 study groups. The 3% of Na CMC with 1% calcium phosphate nanoparticles gel showed that a specific concentration of the nanoparticles with a viscous polymer can cause significant impediment in the drug release rate. The average slopes highlight that the solution has the highest release rate, then the 1% CMC formulations, and lastly the 3% CMC formulations.

TABLE 9
Slope for absorbance versus time graph

| | | 1% CMC | 3% CMC | 1% CMC | 1% CMC | 3% CMC | 3% CMC |
|---|---|---|---|---|---|---|---|
| Sample | Solution | gel | gel | 1% NP | 5% NP | 1% NP | 5% NP |
| 1 | 0.553 | 0.604 | 0.329 | 0.583 | 0.497 | 0.286 | 0.351 |
| 2 | 0.608 | 0.717 | 0.360 | 0.418 | 0.430 | 0.328 | 0.390 |
| 3 | 0.585 | 0.510 | 0.363 | 0.288 | 0.437 | 0.342 | 0.375 |
| 4 | 0.535 | 0.671 | 0.270 | 0.358 | 0.511 | 0.321 | 0.411 |
| 5 | 0.669 | 0.426 | 0.311 | 0.291 | 0.485 | 0.315 | 0.322 |
| 6 | 0.527 | 0.566 | 0.317 | 0.321 | 0.479 | 0.319 | — |
| 7 | — | 0.488 | — | 0.321 | — | — | — |
| 8 | — | 0.485 | — | 0.288 | — | — | — |
| Ave. | 0.579 | 0.557 | 0.325 | 0.358 | 0.473 | 0.319 | 0.370 |

ANOVA was performed on the slopes of each sample from the different preparations (table 10). The results indicate there was a statistically significant difference between the different study groups shown by a p-value <0.001. Also, the F statistic ratio shows variability between the different treatment groups compared to the variability within the groups. The high F ratio indicates statistically significant differences comparing the various preparations. Then, post-hoc analysis was performed using Tukey's test by examining all of the slopes from the samples of the preparations (table 11). The solution formulation of 0.68% timolol maleate was statistically significant from the slower release rate formulations such as formulations with 3% CMC polymer and 1% Na CMC with 1% CaP NP, indicated by p-value of <0.05. Furthermore, the 1% CMC gel which was documented as similar in release rate to the solution was also statistically different than the 3% CMC polymer formulations and 1% CMC with 1% CaP NP, indicated by p-value of <0.001. The solution and 1% CMC gel had two fastest release rate data and was considered greatly different than other formulations except for the 1% CMC 5% CaP NP. The 1% CMC 5% CaP NP gel was the third fastest formulation and was statistically different in comparison to all the 3% CMC gel formulations, p-value <0.05.

TABLE 10
ANOVA of Slopes

| Source | DF | SS | MS | F | P |
|---|---|---|---|---|---|
| Setting | 6 | 0.4768818 | 0.079480297 | 17.404727 | <.0001 |
| Error | 38 | 0.1735305 | 0.004566592 | | |
| Total | 44 | 0.6504123 | 0.014782098 | | |

TABLE 11
Tukey's test of Slopes

| Formulation | Mean difference | q | p-value | 95% CI |
|---|---|---|---|---|
| Solution vs 3% CMC 1% | 0.261 | 9.4606 | <.0001 | 0.1396 to 0.3824 |
| CaP NP | | | | |
| Solution vs 3% CMC gel | 0.2545 | 9.225 | <.0001 | 0.1331 to 0.3759 |
| Solution vs 3% CMC 5% | 0.2097 | 7.2474 | <.0001 | 0.082379 to 0.33702 |
| CaP NP | | | | |
| Solution vs 1% CMC 1% | 0.221 | 7.6959 | <.0001 | 0.094813 to 0.34719 |
| CaP NP | | | | |
| Solution vs 1% CMC 5% | 0.106333 | 3.8543 | 0.1195 | −0.015063 to 0.22773 |
| CaP NP | | | | |
| Solution vs 1% CMC gel | 0.021125 | 0.8186 | 0.9971 | −0.092431 to 0.13468 |
| 1% CMC gel vs 3% CMC | 0.239875 | 9.2952 | <.0001 | 0.12632 to 0.35343 |
| 1% CaP NP | | | | |
| 1% CMC gel vs 3% CMC | 0.233375 | 9.0434 | <.0001 | 0.11982 to 0.34693 |
| gel | | | | |
| 1% CMC gel vs 3% CMC | 0.188575 | 6.9225 | 0.0003 | 0.068706 to 0.30844 |
| 5% CaP NP | | | | |
| 1% CMC gel vs 1% CMC | 0.199875 | 8.3658 | <.0001 | 0.094743 to 0.30501 |
| 1% CaP NP | | | | |
| 1% CMC gel vs 1% CMC | 0.0852083 | 3.3019 | 0.2546 | −0.028347 to 0.19876 |
| 5% CaP NP | | | | |
| 1% CMC 5% CaP NP vs | 0.154667 | 5.6063 | 0.0053 | 0.03327 to 0.27606 |
| 3% CMC 1% CaP NP | | | | |
| 1% CMC 5% CaP NP vs | 0.148167 | 5.3707 | 0.0085 | 0.02677 to 0.26956 |
| 3% CMC gel | | | | |
| 1% CMC w/ 5% CaP NP vs | 0.103367 | 3.5724 | 0.1788 | −0.023955 to 0.23069 |
| 3% CMC w/ 5% CaP NP | | | | |
| 1% CMC w/ 5% CaP NP vs | 0.114667 | 4.4434 | 0.0465 | 0.0011109 to 0.22822 |
| 1% CMC w/ 1% CaP NP | | | | |
| 1% CMC 1% CaP NP vs 3% | 0.04 | 1.55 | 0.9254 | −0.073556 to 0.15356 |
| CMC 1% CaP NP | | | | |
| 1% CMC 1% CaP NP vs 3% | 0.0335 | 1.2981 | 0.9673 | −0.080056 to 0.14706 |
| CMC gel | | | | |
| 1% CMC 1% vs 3% CMC | 0.0113 | 0.4148 | 0.9999 | −0.10857 to 0.13117 |
| 5% CaP NP | | | | |
| 3% CMC 5% CaP NP vs 3% | 0.0513 | 1.773 | 0.8681 | −0.076021 to 0.17862 |
| CMC 1% CaP NP | | | | |
| 3% CMC 5% CaP NP vs 3% | 0.0448 | 1.5483 | 0.9258 | −0.082521 to 0.17212 |
| CMC gel | | | | |
| 3% CMC gel vs 3% CMC | 0.0065 | 0.2356 | 1 | −0.1149 to 0.1279 |
| 1% CaP NP | | | | |

*bold indicates statistically significant results indicated by p-value <0.05.

The release rate data for each preparation using the time and percent absorbance was used to curve fit each sample. The Sigmoid curve fit was the model that best fit the scatter plot graph. The equation for this curve has 4 variables, m1, m2, m3, and m4. The averages were taken for each sample from the preparations and are listed on table 12.

TABLE 12
Sigmoid Curve Fit Data

| | | | | | | | 3% CMC |
|---|---|---|---|---|---|---|---|
| | | 1% CMC | 3% CMC | 1% CMC | 1% CMC | 3% CMC | 5% CaP |
| | Solution | gel | gel | 1% CaP NP | 5% CaP NP | 1% CaP NP | NP |
| m1 | 114.4783 | 109.035 | 108.1267 | 111.66 | 105.8 | 106.7267 | 109.82 |
| m2 | 2.379183 | 1.257518 | 0.5899 | 0.559111 | 0.80071 | −0.24231 | 1.117284 |
| m3 | 116.6945 | 129.1526 | 194.065 | 209.1309 | 125.9383 | 191.9133 | 154.9963 |
| m4 | 1.346883 | 1.474459 | 1.394717 | 1.305538 | 1.599817 | 1.399767 | 1.424283 |

The viscosity of each preparation was determined. 3% CMC with 1% CaP NP gel was the most viscous follow by 3% CMC gel, 3% CMC with 5% CaP NP gel, 1% CMC with 1% CaP NP gel, 1% CMC with 1% CaP NP gel, and 1% CMC gel, respectively. These results did not correlate with what was expected as viscosity of the different gels. The viscosity data does coincide with the release rate studies for the gel preparations.

The viscosity data obtained for each gel was used in developing curve fit analysis using Bingham and Ostwald-De Waele model (table 13). The flow consistency index (k) and the flow behavior index (n) were assessed for the different graphs using the Ostwald-De Waele model. The n values of all the gels were <1 which indicated that the gels displayed pseudoplastic behavior. This behavior highlights that the gels displayed lower viscosity at higher shear rates which was as predicted. As previously discussed, Na CMC polymers with higher molecular weights display greater shear-thinning properties compared to smaller molecular weight CMC polymers.⁹ This correlates with what was seen with the n values between the 1% CMC gel and 3% CMC gel even with the addition of CaP NP. The k values from the Ostwald-De Waele model were also consistent with the release rate studies. The slowest gel release rate was 3% CMC with 1% CaP NP which had the highest k value, followed by 3% CMC gel, and 3% CMC with 5% CaP NP, respectively. The 1% CMC gel formulations displayed the lower k values.

Furthermore, thixotropy was determined which illustrated hysteresis loops and shear thinning properties which varied in each of the gel formulations especially when CaP NP were added. A flow curve determined two values for stress, one value for increased rate of shearing and the other for decreasing rate of shear and difference of these two values noted on table 13. The less viscous polymer gels of 1% CMC displayed negative values which was unseen with the 1% CMC gel without any nanoparticle addition. The more viscous polymers of 3% CMC with nanoparticles added displayed smaller values compared to the 3% CMC gel by itself. Also, the yield value was also consistent with the release rate studies results. The slowest release rate of all the gels was the 3% CMC with 1% CaP NP which was also the gel that displayed the highest yield value. The 3% CMC gel formulations displayed higher yield values compared to the 1% CMC gel formulations indicating there was a higher resistance with the 3% CMC gel formulations to flow under stress. There was no linear relationship between the values of the different rheology parameters assessed besides the yield values. The yield values of 3% CMC gel formulations displayed a linear relationship with a R² value of 0.996 and p-value <0.001.

TABLE 13
Rheology Parameters

| | 1% CMC | 3% CMC | 1% CMC | 1% CMC | 3% CMC | 3% CMC 5% |
|---|---|---|---|---|---|---|
| | gel | gel | 1% CaP NP | 5% CaP NP | 1% CaP NP | CaP NP |
| K | 2.827 | 164.80 | 1.887 | 4.376 | 183.30 | 162.50 |
| N | 0.5334 | 0.2308 | 0.5272 | 0.4673 | 0.2212 | .2101 |
| Yield Value | 19.17 | 477.40 | 19.34 | 35.46 | 506.50 | 427.40 |
| Thixotropy | 1903 | 4194 | −746.05 | −973.5 | 115.15 | 105.10 |

### Discussion

Timolol maleate release rates for each preparation varied based on viscosity of the polymers. The 1% CMC gel was first studied and results showed minimal difference in release rate compared to the solution. The 3% CMC polymer gel displayed longer release rate than the 1% CMC polymer gel highlighting that viscosity was a big component in active drug release. Viscosity has previously proven to have a correlation with delaying drug release; however, the novel approach of incorporating calcium phosphate nanoparticles also displayed properties that slowed release rates of timolol maleate. This delay of release rate was dependent upon the concentration of the nanoparticles. It was expected that the 5% calcium phosphate nanoparticles would have further delayed release compared to 1% calcium phosphate nanoparticles, however, the 5% calcium phosphate nanoparticles caused a quicker release of the active drug. This phenomenon was seen with both concentrations of Na CMC polymer of 1% and 3%. Although there are no previous studies with calcium phosphate nanoparticles conducted to assess if this concentration dependent activity is seen with other polymers or formulations, it is evident that there is an interaction with the Na CMC polymer and nanoparticles. This interaction is seen with higher concentrations of calcium phosphate nanoparticles despite the rest of the formulation's components. The 1% calcium phosphate nanoparticles resulted in the slowest release rate which also highlights that the nanoparticles help delay release rate compared to a viscous polymer alone.

Analysis of variance was performed which showed the average slopes of several the formulations were statistically significant. Post-hoc Tukey's test displayed that the formulations had variations in release rate. The results coincided with the release rate data highlighting that the solution had the fastest release compared to all of the gels. Furthermore, the 1% CMC gel and 1% CMC gel with 5% calcium phosphate nanoparticles were the fastest after the solution and were statistically different than the other gel formulations. These results support the trend that the calcium phosphate nanoparticles are concentration dependent on their ability to slow release rates. This could be attributed to the possible interaction of the higher concentrations the nanoparticles with the polymer. Also, the 3% CMC polymers were statistically different than the 1% CMC polymer formulations even with inclusion of nanoparticles. The solution versus the 1% CMC gel were not statistically significant, however, the addition of 1% CaP NP to the 1% polymer exhibited significant difference in release rate. Furthermore, the 3% CMC polymer versus the solution slowed down the release rate and the addition of 1% CaP NP to the 3% CMC polymer even further delayed the release. This highlights the notion that CaP NP cause an additional delay in release rate regardless of the viscosity of the polymer.

The Sigmoid curve-fit model showed the various m values which were plotted. The results showed that the different preparations when directly compared versus time are consistent with the release rates of all the samples of each gel and solution. The m3 and m4 values determine the release rate and the shape of the curve. However, the m3 values do not consistently correlate with the data found in this study which could be attributed to the m4 changes in the shape of the curve. Nonetheless, when the average m1-m4 values were graphed, the results did illustrate the same trend as the release rate studies.

The viscosity data showed that the 3% CMC gel displayed the most viscosity compared to the 1% CMC gel as expected. However, the calcium phosphate polymers at varying concentrations displayed different viscosities and the concentration of Na CMC polymer was also a factor. For example, the 3% polymer gel formulations with the 1% calcium phosphate nanoparticles had the most viscosity compared to all the other 3% CMC gel formulations; conversely, the 1% polymer gel formulations with 5% calcium phosphate nanoparticles was the most viscous compared to all the 1% CMC gel formulations. This also signifies that the interaction between the nanoparticles and polymer are concentration dependent for both components. The thixotropy of the gel formulations varied when direct comparisons were made. The gels without any nanoparticle addition demonstrated results that were expected from previous studies of Na CMC polymer. However, the 1% CMC gel with calcium phosphate nanoparticles showed a negative value indicating that the viscosity of the gel increased with shear stress which is usually not seen with CMC polymer alone. The 3% CMC gel with calcium phosphate nanoparticles displayed smaller positive values which indicate that viscosity did decrease with time-dependent shear stress but at a lower value amount than the polymer alone. There seems to be a significant difference in thixotropy based on the polymer and the nanoparticles. When the higher concentration of the polymer is used, it supersedes the effects of the polymer evident by the positive thixotropic values seen. However, with the lower concentration of the polymer, the nanoparticles cause an effect that overwhelms what is normally seen and the viscosity of the gel is increased in a time-dependent manner.

The Ostwald-De Waele model showed that all the gels maintained psuedoplastic behavior which was expected due to the properties of high molecular weight Na CMC polymer. The calcium phosphate nanoparticles did not change the pseudoplasticity of the gel which is beneficial for ocular drug delivery and patient comfort. The flow consistency index and yield value obtained from the Bingham model seemed to correlate with each other overall in the same general trend. The yield value was seen higher with the more viscous 3% CMC gel formulations than the 1% CMC gel formulations. The 3% CMC with 1% calcium phosphate nanoparticles has the highest yield value followed by the 3% CMC gel. However, with the 1% CMC gel preparations, the 1% CMC with the 5% calcium phosphate nanoparticles had the highest yield values followed by the 1% CMC with 1% calcium phosphate nanoparticles. This indicates that calcium phosphate nanoparticles play a role in initial resistance of the gel to flow but the extent of this behavior is unknown. This same behavior is seen with the flow consistency index which again distinguishes that the polymer and nanoparticles have an interaction that is dependent upon concentration and that interaction determines the rheological behavior of the whole gel. Also, the properties displayed of the gels are dependent upon the concentrations of both the polymer and nanoparticles to determine which agent is dominant and it determines the properties of the gel.

### Conclusion

This study illustrated the effect of the viscosity and addition of calcium phosphate nanoparticles with the drug delivery rate of timolol maleate. Previous studies have shown that ophthalmic gels delay release of active drug versus solutions. However, calcium phosphate nanoparticles addition was previously not studied and did show a concentration dependent delay in drug release rate. The 1% calcium phosphate nanoparticles displayed the slowest release rate which warrants further study of nanoparticles in ophthalmic formulations without compromising ocular comfort. As previously discussed, ocular discomfort can occur with increased viscosity which concludes that even though a more viscous polymer is ideal, it is not practical. The use of calcium phosphate nanoparticles can exhibit similar pharmaceutical profile as a polymer in drug delay. Also, the pseudoplastic characteristics of the gel were not compromised with the nanoparticles which allows for shear-thinning properties and prevents ocular discomfort. This study concludes that further studies are indicated to assess the concentration of calcium phosphate nanoparticles when used for ophthalmic gels in conjunction with polymers. The results of this study imply that there is an indubitable correlation between the polymer and calcium phosphate nanoparticles at varying concentrations of both. Finding the appropriate ratio between the two components can allow for optimal drug delivery and mitigation of patient discomfort.

### Example 1 and 2 Summary of Results and Data

Ten centimeter segments of cellulose ester dialysis membrane (MWCO of 3,500-5,000 D) were washed with deionized water to remove the preservative. The dialysis membrane segments were then equilibrated with DPBS for 24 hours or longer at ambient temperature. The dialysis tubing was closed at one end, filled with approximately 1 g of preparation, and then the top end was clamped. Each filled dialysis membrane “bag” was placed in a beaker filled with 100 mL of DPBS to elicit sink conditions. A stir bar was placed in the beaker that was then covered with parafilm. Each sample “set up” was then placed on a multi-station stir plate at ambient temperature and agitated at the same speed. The weight was documented for each set up and deionized water was added, if needed, to compensate for water loss. Timed Timolol/DPBS samples were returned to the set up after measurement using dedicated disposable UV cuvettes and transfer pipettes. Therefore, the DPBS receiving fluid in each set up was not diluted by adding DPBS in order to compensate for measured samples being set aside.

Timolol was released from the preparation and diffused through the dialysis membrane into DPBS. The high molecular weight polymers and the CaP NPs remained within the dialysis bag. The absorbance (295 λ) of Timolol was measured at regular timed intervals using a UV-Vis Spectrophotometer. Release data was collected for the solution for 7 hrs or more and was collected for the gels for at least 18 hrs or more. Fifteen or more sampling times were performed for each experimental run with at least six runs being conducted for each preparation. The actual amount of gel preparations transferred to the dialysis bag was measured and absorbance readings were normalized to 1 g of gel being tested. The measured amounts of released Timolol were expressed as percentages of the total amount of Timolol that can be theoretically released (% Theory). That is, percent theory equals 100 times the measured amount of Timolol divided by 0.067 mg/ml (1 gm of 0.68% Timolol Maleate diluted to 101 ml).

Timolol concentrations, expressed as % Theory, were plotted for each experimental run versus the time at which the samples were measured. The data were assessed using a sigmoid curve fit using the general form of a logistic function (KaleidaGraph). The four variable equation used in the Sigmoidal curve fitting of the data as defined in KaleidaGraph is: Y=M1+(M2-M1)/(1+(X/M3)̂M4). In this paper, Y is the amount of Timolol released (% Theory) at X (time in minutes). One variable of the logistic function corresponds to the concentration before the dialysis run begins (M2). The curve fitting was performed with this variable fixed at 0.0% theory. A second variable (M1) corresponds to the concentration at time infinity. This variable was defined as 100% Theory during the curve fitting with the assumption that all Timolol will be released from all preparations by infinity. The data was also fit to this equation with the variables M1 and M2 not being defined (100 & 0.0) and allowed to vary. The averages of the variable values for M1 and M2 were 98.8±5.5 and 1.0±0.9, respectively. The very slight improvement in the r2 value seen when M1 and M2 are left to vary in value did not justify the loss in degrees of freedom.

The M3 parameter was allowed to vary with M2 and M1 fixed. It is the time at which the midpoint (50% theory) or point of inflection is reached between the lowest (0.0%) and highest amounts (100%) of Timolol release. Thus, the value for M3 is an effective measure of how quickly Timolol is released from the preparation and then diffuses through the membrane into the receiving fluid (DPBS). The fourth variable (M4) was allowed to vary and is considered a shape parameter. It gives less information as to how rapidly Timolol is being released as compared to M3.

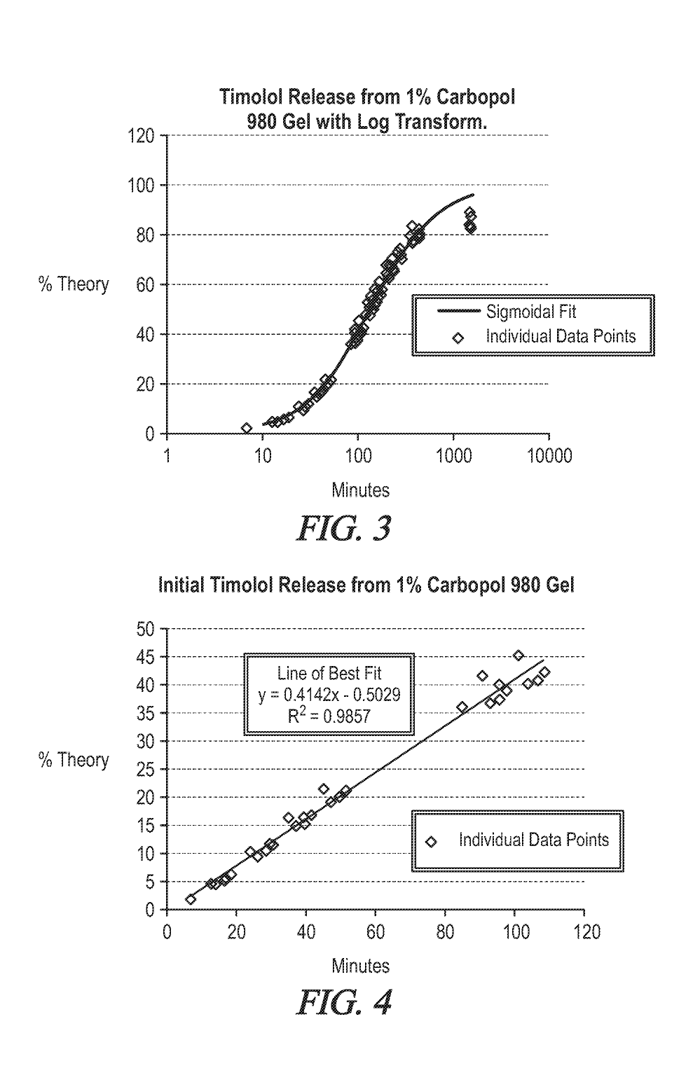

A representative sample of a typical Timolol release profile is shown in FIG. 2 with a sigmoidal fit along with the individual data points (85 pairs) from the 1% C980 experimental runs. The sigmoidal nature of the fit is more easily recognized if the fit and data are shown with log (time) rather than time as the X-axis (FIG. 3).

Observing FIGS. 2 and 3; the data collected near 75% Theory is in the upper curvature of the S-shaped curve (departing from the initial linear portion), but is still closely described by the sigmoid model. Therefore, the time to reach 75% Theory (T75) was calculated for each individual fit using the equation: 75=M3× [symbol] (3) [symbol] ̂(1/M4) , in which the assumptions of 0% Theory at time zero and 100% Theory at infinity are applied.

The linear portion of the curve was examined as a measure of the rate of release. The initial data pairs were fit using linear regression. The selection of data pairs was made visually along with an examination as to an increase or reduction in the r2 value. Higher slope values are considered indicative of faster release rates of Timolol (FIG. 4).

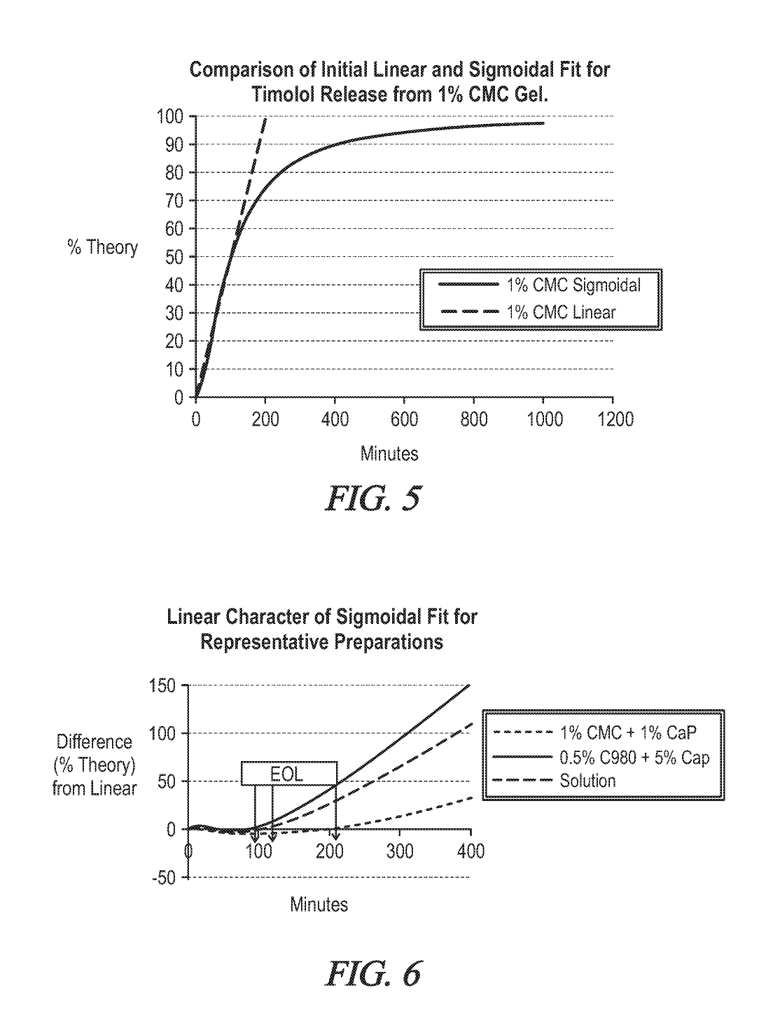

Another approach to examining the linearity of the release is to assign a lag time of two minutes and to define the initial “linear” portion of the fitted release curve as occurring up to the time defined by the M3 value. FIG. 5 shows an example of this approach and the sigmoidal fit is nearly linear in form up to about 50% Theory.

Subtracting the fitted sigmoidal value from the calculated “linear” values at each time point gives an indication of the “linearity” of the sigmoidal fit. That is, the closer the difference is to zero the more linear that portion of the sigmoidal fit is. As seen in Figure below, it is apparent that the 1% CMC plus 1% CaP NP gel is somewhat linear (differences of ±5% or less) for a longer period of time than the 0.5% Carbopol 980 plus 5% CaP gel. The “end of linearity” (EOL) is defined as the last time point (minutes) in which the difference from linearity is close to zero.

The M3, M4, and T75 values were determined using a sigmoidal fit for each experimental run. Prior to statistical analysis, any outliers for the different preparation's M3 values were detected using Dixon-type tests. Outlier experimental runs were then discarded in further statistical analysis. The generated M3, M4, and T75 values were treated as independent data points and one way ANOVA and Tukey's post-hoc tests (p<0.05) were used to determine statistical significance for M3, M4, and T75 values of the different preparations. The average values of the individual M3, M4, and T75 values for each of the preparations are treated as representative of that preparation.

Gel viscosities were assessed using a Haake Viscotester 550. Curve-fit analysis was performed using the Ostwald-De Waele and Bingham models. The Ostwald-De Waele model allowed for the viscosity related constant (k) and flow behavior index (n) to be determined according to the equation:T=kγ·̂n, where T is the shear stress. The higher the k value, the more viscous the gel preparation is. Fluids are considered to be Newtonian if n is equal to 1. Fluids are considered to be pseudoplastic (shear thinning) if n is less than 1 and considered more shear thinning as the value for n decreases. The Bingham model allowed for the calculation of the yield value for each gel preparation.

A nonlinear curve fit (sigmoid model—KaleidaGraph) was used to evaluate each experimental run for % Theory versus sampling time. The R2 values that were obtained for all the individual curve fits ranged from 0.96 to 0.99 with an average value of 0.99. The M3, M4, and T75 values for each experimental run were averaged and are listed in Table 14 and 15. Larger M3 midpoint values indicate slower Timolol release rates. The M4 variable is a shape parameter; but in general, an increase in M4 in the range seen in this study results in a faster rate at which 90% Theory is reached. Larger T75 values are an indicator of slower release.

TABLE 14
Curve Fit Data for CMC Gels

| | | | | | | | 3% |
|---|---|---|---|---|---|---|---|
| | | | | | | | CMC + |
| | | 1% CMC | 3% CMC | 1% CMC + | 1% CMC + | 3% CMC + | 5% |
| | Sol. | gel | gel | 1% CaP | 5% CaP | 1% CaP | CaP |
| Ave. | 102.44 | 100.99 | 153.66 | 195.75 | 121.67 | 173.58 | 148.69 |
| M3 | | | | | | | |
| Ave. | 1.390 | 1.573 | 1.431 | 1.213 | 1.624 | 1.423 | 1.420 |
| M4 | | | | | | | |
| Ave. | 228.64 | 220.79 | 349.18 | 503.10 | 241.34 | 370.39 | 294.14 |
| T75 | | | | | | | |

TABLE 14
Curve Fit Data for C980 Gels

| | | | | | | 0.5% | 1.0% |
|---|---|---|---|---|---|---|---|
| | 0.5% | 1% C980 | 0.5% C980 + | 0.5% C980 + | 0.5% C980 + | C980 + 5% | C980 + 1% |
| | C980 gel | gel | 0.5% CaP | 1% CaP | 2% CaP | CaP | CaP |
| Ave. | 101.38 | 134.26 | 115.64 | 153.49 | 87.97 | 81.96 | 154.42 |
| M3 | | | | | | | |
| Ave. | 1.290 | 1.262 | 1.647 | 1.346 | 1.705 | 1.583 | 1.450 |
| M4 | | | | | | | |
| Ave. | 277.38 | 320.93 | 226.95 | 356.78 | 168.41 | 165.43 | 331.17 |
| T75 | | | | | | | |

ANOVA results indicated that there were statistically significant differences between all of the preparation values that were considered indicators of Timolol release rate (M3, M4, & T75 at a p value <0.0001. Tukey's post-hoc analysis of M3, M4, and T75 values was used to determine which preparations generated Timolol release rates that were statistically significant from each other. Overall, a difference of 66 min (one pair was significantly different with a difference of 58 min) in the average M3 value between the pair of preparations being compared is indicative of statistical significance. Of the many significant differences in Timolol release rates between the preparations, the most relevant differences are displayed in Table 16.

TABLE 16
Post Hoc Tukey's Test of Experimental M3 Fit Values

| | | Differences | |
|---|---|---|---|
| Preparation One - | Preparation Two - | between Mean M3 | |
| Slower Release | Faster Release | Values (min.) | P value |
| 1% CMC + 1% CaP | 1% CMC | 94.76 | <0.0001 |
| 1% CMC + 1% CaP | 0.5% C980 | 93.31 | <0.0001 |
| 1% CMC + 1% CaP | 1% CMC + 5% CaP | 74.20 | 0.004 |
| 0.5% C980 + | 0.5% C980 + 5% | 71.53 | 0.01 |
| 1% CaP | CaP | | |
| 0.5% C980 + | 0.5% C980 + 2% | 65.52 | 0.03 |
| 1% CaP | CaP | | |

TABLE 17
Post Hoc Tukey's Test of Experimental M4 Fit Values

| | | Differences | |
|---|---|---|---|
| Preparation One - | Preparation Two - | between Mean M4 | |
| Slower Release | Faster Release | Values | P value |
| 1% CMC + 1% CaP | 1% CMC | −0.3598 | 0.03 |
| 1% CMC + 1% CaP | 1% CMC + 5% CaP | −0.4112 | 0.01 |
| 0.5% C980 | 0.5% C980 + 2% | −0.4145 | 0.02 |
| | CaP | | |

TABLE 18
Post Hoc Tukey's Test of Experimental T75 Fit Values

| | | Differences | |
|---|---|---|---|
| Preparation One - | Preparation Two - | between Mean T75 | |
| Slower Release | Faster Release | Values (min.) | P value |
| 1% CMC + 1% CaP | 1% CMC | 282.31 | <0.0001 |
| 1% CMC + 1% CaP | 0.5% C980 | 225.72 | 0.0037 |
| 1% CMC + 1% CaP | 1% CMC + 5% CaP | 261.77 | 0.0003 |
| 0.5% C980 + | 0.5% C980 + 5% | 191.35 | 0.044 |
| 1% CaP | CaP | | |
| 0.5% C980 + | 0.5% C980 + 2% | 188.37 | 0.0513 |
| 1% CaP | CaP | | |

An excellent correlation (r²=0.8526) was achieved in the anticipated order when the values of the linear slopes (initial portion of release) were regressed against the average M3 values. It appears that the M3 value adequately describes the release rate for the initial portion of the data. An excellent positive correlation was obtained between the M3 values for the preparations and EOL. Thus, those preparations which had the slowest time to 50% theoretical release also displayed “linear” behavior for the longest time.

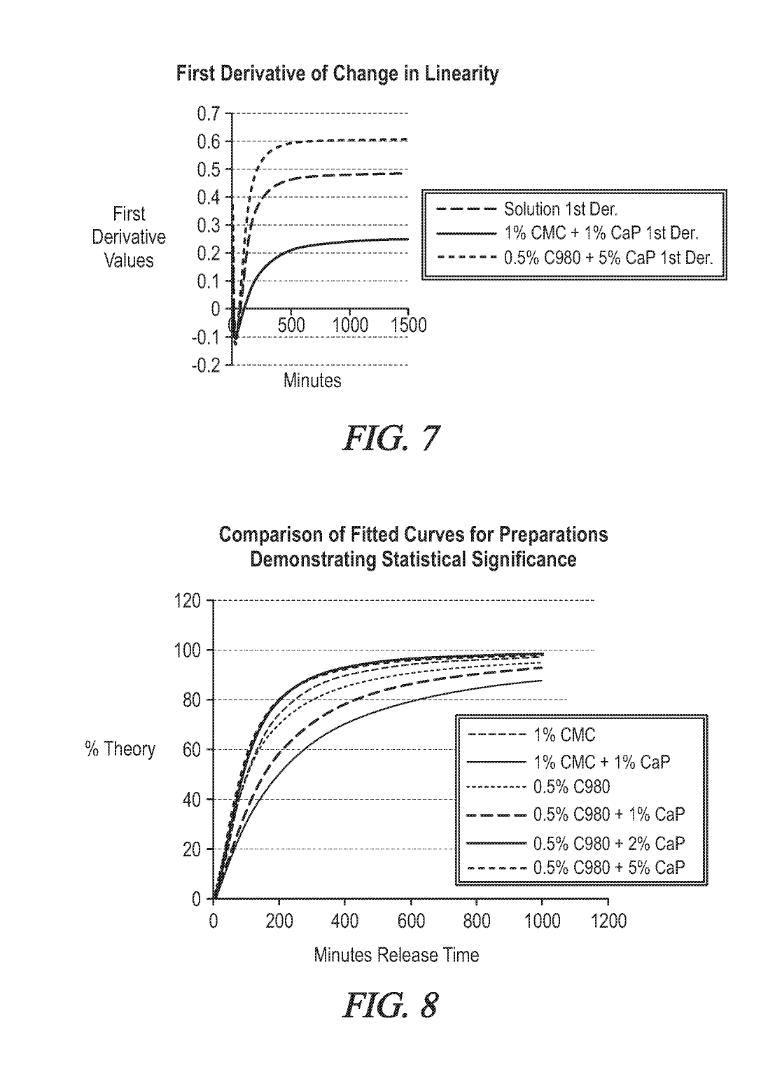

It is possible to observe the rate at which the sigmoidal fit varies from linear character by plotting the first derivative of the difference from linear values versus time. As shown in FIG. 7, a nearly constant level of change is achieved at that time at which 100% Theory is nearly achieved in the sigmoidal model. The nearly constant levels of change are strongly correlated (negatively) with the M3 values indicating that the slope of the linear portion is smaller for those preparations showing a delay in release.

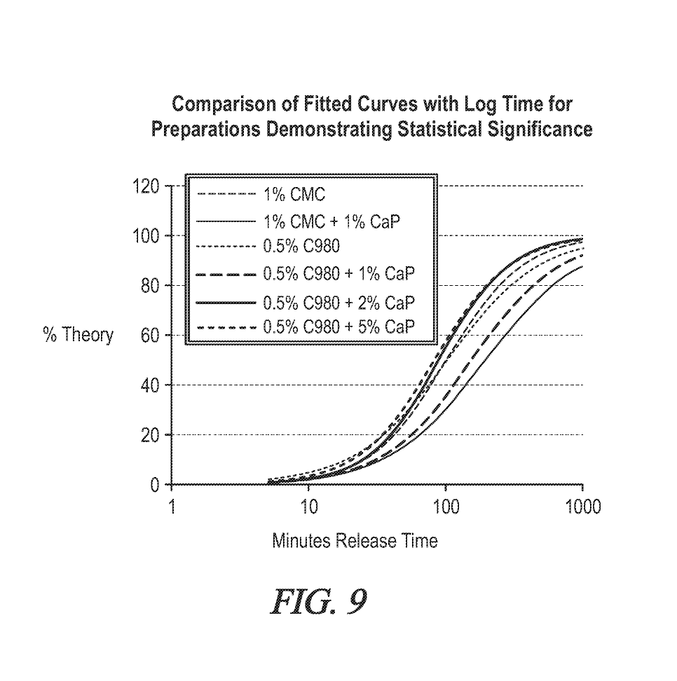

FIGS. 8 and 9 depict the comparison of fitted curves for several preparations in terms of minutes of release time and log time, respectively.

Average T75 values show a strong correspondence with the average M3 values. A correlation coefficient (r) of 0.947 was achieved when the T75 and M3 were regressed against each other. The statistical results for T75 are summarized in Table 17 and in general, a difference of 188 minutes is needed in order to obtain statistical significance. The summarized results for T75 are basically the same as those seen when M3 was used as the indictor of Timolol release rate.

An alternate approach to defining the amount of Timolol released is to fit the data according to the percentage of the last value measured rather than % Theory. The last value was usually obtained after more than 14 hours of release and was referred to as % Overnight. An excellent correlation was achieved when the M3 values fit for the data expressed as % Overnight were regressed versus to the M3 values obtained using % Theory (R²=0.98, slope of 1.02). The M3 value appears to indicate the rate of release in a manner that is fairly robust in regard to how the fit parameters are defined.

Although M3 (midpoint of curve) is considered the best indicator of release rate, the M4 variable (shape) parameter gives some indication as to the slope of the linear portion of the Sigmoid curve. When the M4 value is larger, then the time at which 90% theory is achieved occurs more rapidly. When the average M3 and M4 values were regressed against each other a fair correlation was achieved with a correlation coefficient of 0.6 that is statistically significant. The regression analysis indicates that, in general, a higher M4 value corresponds with a lower M3 value (faster release). There is a statistically significant difference for 0.5% C980 gel M4 values versus 0.5% C980 with 2% CaP M4 values that is not seen with the M3 values. However, the M3 values for these preparations are in the same order in regard to rate of release as seen with the M4 values.

What is claimed is:

1. A pharmaceutical composition comprising
a hydrogel and
calcium phosphate nanoparticles.

2. The composition of claim 1, wherein the hydrogel comprises polymers having functional groups selected from the group consisting of carboxylate, carbonyl, amine and mixtures thereof.

3. The composition of claim 2, wherein the hydrogel comprises hyaluronic acid, alginate, carbopol (cross-linked polyacrylate), sodium alginate, carboxymethyl cellulose, and mixtures thereof.

4. The composition of claim 3, wherein the hydrogel comprises carboxymethyl cellulose or carbopol.

5. The composition of any one of claims 1 to 4, wherein the number ratio of C to P in the calcium phosphate nanoparticles ranges from 1:1 to 3:1.

6. The composition of any one of claims 1 to 5, wherein the calcium phosphate is tricalcium diphosphate.

7. The composition of any one of claims 1 to 5, wherein the calcium phosphate is dibasic calcium phosphate dihydrate.

8. The composition of any one of claims 1 to 7, wherein the calcium phosphate nanoparticles have a size ranging from about 5 to about 200 nm.

9. The composition of claim 8, wherein the calcium phosphate nanoparticles have a size ranging from about 10 to about 100 nm.

10. The composition of claim 9, wherein the calcium phosphate nanoparticles have a size ranging from about 10 to about 80 nm.

11. The composition of any one of claims 1 to 10, wherein the calcium phosphate particles have a surface area ranging from about 10 to about 100 m²/gm.

12. The composition of claim 11, wherein the calcium phosphate particles have a surface area ranging from about 30-60 m²/gm.

13. The composition of any one of claims 1 to 12, wherein the composition comprises about 0.1% to about 5% by weight of the calcium nanoparticles.

14. The composition of claim 13, wherein the composition comprises about 0.5% to about 5% of the calcium nanoparticles by weight of the composition.

15. The composition of claim 13, wherein the composition comprises about 0.5% to about 2% of the calcium nanoparticles by weight of the composition.

16. The composition of any one of claims 1 to 15, wherein the composition comprises about 0.2% to about 5% of polymers having functional groups selected from the group consisting of carboxylate, carbonyl, amine and mixtures thereof by weight of the composition.

17. The composition of claim 16, wherein the hydrogel comprises about 0.5% to about 2% of polymers by weight of the composition.

18. The composition of any one of claims 1 to 17, wherein the composition further comprises a pharmaceutical agent.

19. A method of administering a pharmaceutical agent to a patient in need thereof comprising:
providing the composition of claim 18 and
administering the composition to the patient.

20. The method of claim 19, wherein the administration is topical, ophthalmic, oral, rectal or vaginal.
